(19) United States
(12) Patent Application Publication
(10) Pub. No.: US 2017/0189553 A1
(43) Pub. Date: Jul. 6, 2017

(54) POLYMERS, SYSTEMS, AND METHODS FOR USING AND MONITORING POLYMERS FOR USE IN MEDICAL POLYMERS, IMPLANTS, AND PROCEDURES

(71) Applicant: CANARY MEDICAL INC., Vancouver (CA)

(72) Inventor: William L. Hunter, Vancouver (CA)

(73) Assignee: CANARY MEDICAL INC., Vancouver, BC (CA)

(21) Appl. No.: 15/320,292

(22) PCT Filed: Jun. 25, 2015
(86) PCT No.: PCT/US2015/037828

§ 371 Date: Dec. 19, 2016

Related U.S. Application Data
(60) Provisional application No. 62/017,159, filed on Jun. 25, 2014.

(51) Int. Cl. A61K 49/00; A61L 29/14; A61L 17/00; H04Q 9/00; A61L 31/14; A61K 47/30; G01N 33/44; H04B 5/00; A61L 29/04; A61L 31/04

(52) U.S. Cl. CPC A61K 49/00; A61L 29/04; A61L 29/14; A61L 17/00; A61L 31/04; A61L 31/14; A61K 47/30; G01N 33/442; H04B 5/0068; H04Q 9/00; H04Q 2209/40; H04Q 2209/82; G06F 19/322

(57) ABSTRACT

A polymer comprising a medical polymer and one or more sensors positioned within or upon said medical polymer. The sensor may be selected from the group consisting of fluid pressure sensors, contact sensors, position sensors, pulse pressure sensors, liquid volume sensors, liquid flow sensors, chemistry sensors, metabolic sensors, accelerometers, mechanical stress sensors and temperature sensors. The medical polymer may be a biodegradable polymer or a non-biodegradable polymer.

### CROSS-REFERENCE TO RELATED APPLICATION

This application claims the benefit under 35 U.S.C. §119(e) of U.S. Provisional Patent Application No. 62/017,159, filed Jun. 25, 2014, which application is incorporated herein by reference in its entirety.

### FIELD OF THE INVENTION

The present invention relates generally to polymers, and more specifically, to polymers that are suitable for use as and in a wide variety of medical implants, medical devices and medical procedures.

### BACKGROUND

Polymers are large molecules (or macromolecules) which are composed of repeated subunits, or monomers. They have a broad range of properties (e.g., toughness, viscoelasticity, melting point) and can be utilized as and in a wide variety of medical implants, medical devices and medical procedures.

Typically, polymers are commonly classified as synthetic (i.e., artificially manufactured), or non-synthetic (i.e., naturally occurring). Polymers may also be classified in other ways as well (e.g., biodegradable or non-biodegradable, swellable or non-swellable). As will be evident to one of skill in the art however, many polymers can have more than one property. For example, a medical polymer or implant may be composed of both synthetic and non-synthetic polymers, and be only partially biodegradable.

Polymers have been utilized for decades in medicine, and more recently are commonly utilized in almost all medical polymers and implants. Representative examples include catheters (which can be composed of a wide variety of polymers such as polyurethanes, polyamides, polyolefins, polyvinylchloride (PVC), polyimides, and polyetheretherketones (or PEEK), vascular grafts (e.g., polytetrafluorethylene or “PTFE”), meshes (e.g., polylactic acid or PLA), drug delivery polymers (e.g., PLA, poly (lactic-co-glycolic) acid “PLGA”, and polycaprolactone “PCL”), and bone cements (e.g., poly (methyl methacrylate) “PMMA”).

Polymers however are susceptible to a number of difficulties when utilized in the context of medical applications. For example: 1) they can be susceptible to biofilm formation and subsequent infection; 2) breaking or fracture and subsequent implant or polymer failure; 3) wearing, and subsequent polymer or implant failure; and 4) clogging.

The present invention discloses medical polymers having sensors that can be utilized to diagnose, predict, and overcome previous complications and difficulties, and further provides other related advantages.

### SUMMARY

Briefly stated, a wide variety of polymers are provided with a number of sensors to monitor the integrity and efficaciousness of the polymer (whether utilized alone, or as or with another medical device or implant). Polymers of the present invention can be formed into a vast array of shapes and sizes, which in preferred embodiments are suitable for medical applications. Representative examples of polymer forms include solid forms such as films, sheets, molded, cast, or cut shapes. Other solid forms include extruded forms which can be made into tubes (e.g., shunts, drainage tubes, and catheters), and fibers which can be knitted into meshes or used to make sutures. Liquid forms of polymers include gels, dispersions, colloidal suspensions and the like. Particularly preferred polymers for use within the present invention are medical polymers, e.g., polymers which are provided in a sterile and/or non-pyrogenic form, and suitable for use in humans. Representative examples of polymers include polyester, polyurethanes, silicones, epoxy resin, melamine formaldehyde resin, acetal, polyethyelene terephthalate, polysulphone, polystyrene, polyvinyl chloride, polyamide, polyolefins, polycarbonate, polyethylene, polyamides, polimides, polypropylene, polytetrafluoroethylene, ethylene propylene diene rubber, styrenes (e.g., styrene butadiene rubber), nitriles (e.g., nitrile rubber), hypalon, polysulphide, butyl rubber, silicone rubber, cellulose, chitosan, fibrinogen, collagen, hyaluronic acid, PEEK, PTFE, PLA, PLGA, PCL and PMMA.

Within one embodiment, sensors can be positioned (depending of course on the physical form of the polymer) on the surface of, on top (or bottom, or side) of, within or inside of the polymer. When the phrase “placed in (or on) a polymer” (or “medical polymer) is utilized, it should be understood to refer to any of the above embodiments, unless the context of the usage implies otherwise. Within certain embodiments, the sensors are of the type that are passive and thus do not require their own power supply.

A wide variety of sensors can be utilized within the present invention, including for example, fluid pressure sensors, contact sensors, position sensors, accelerometers, vibration sensors, pulse pressure sensors, liquid (e.g., blood) volume sensors, liquid (e.g., blood) flow sensors, liquid (e.g., blood) chemistry sensors, liquid (e.g., blood) metabolic sensors, mechanical stress sensors, and temperature sensors. Within other embodiments the one or more sensors can be a wireless sensor, and/or a sensor that is connected to a wireless microprocessor.

Within particularly preferred embodiments a plurality of sensors are positioned on the polymer, and within yet other embodiments more than one type of sensor is positioned on the polymer. Within other related embodiments the plurality of sensors are positioned on or within the polymer at a density of greater than 1, 2, 3, 4, 5, 6, 7, 8, 9, 10 or 20 sensors per square centimeter. Within other embodiments the plurality of sensors are positioned on or within the polymer at a density of greater than 1, 2, 3, 4, 5, 6, 7, 8, 9, 10 or 20 sensors per cubic centimeter. Within either of these embodiments there can be less than 50, 75, 100, or 200 sensors per square centimeter, or per cubic centimeter.

Within other embodiments of the invention each medical polymer has a unique device identification number. Within further embodiments one or more (or each) of the sensors have a unique sensor identification number. Within yet other embodiments one or more (or each) of the sensors is uniquely defined within a specific position on or within the polymer. Within other embodiments one or more sensors are placed randomly on or within the polymer.

According to various embodiments, sensors are placed at different locations in a polymer in order to monitor the operation, movement, medical imaging, function, wear, performance, potential side effects, medical status of the patient and the medical status of the polymer and its interface with the live tissue of the patient. Live, continuous, in situ, monitoring of patient activity, patient function, polymer activity, polymer function, polymer patency, performance, placement, surface characteristics (flow and chemical content of fluids moving over or through a surface of the polymer); presence of inflammatory tissues, bacteria or biofilm on the surface etc.), polymer forces and mechanical stresses, polymer and surrounding tissue anatomy (imaging), mechanical and physical integrity of the catheter, and potential side effects is provided. In addition, information is available on many aspects of the polymer and its interaction with the patient's own body tissues, including clinically important measurements not currently available through physical examination, medical imaging and diagnostic medical studies.

According to one embodiment, the sensors provide evaluation data of any motion, movement and/or migration of the polymer during and after placement. Motion sensors and accelerometers can be used to accurately determine the movement of the medical polymer during physical examination and during normal daily activities between visits. Motion sensors and accelerometers can also be used to accurately determine the movement of the medical polymer during placement by the physician.

According to another embodiment, contact sensors are provided between the medical polymer) and the surrounding tissue. In other embodiments, vibration sensors are provided to detect the vibration between the medical polymer and the surrounding tissue. In other embodiments, strain gauges are provided to detect the strain between the polymer and the surrounding tissue. Sudden increases in strain may indicate that too much stress is being placed on the polymer, which may increase damage to the surrounding body tissues or even result in perforation of the body lumen that is being instrumented.

According to other embodiments, accelerometers are provided which detect vibration, shock, tilt and rotation. According to other embodiments, sensors for measuring surface wear, such as contact or pressure sensors, may be embedded at different depths within the polymer in order to monitor contact of the catheter with vessel walls, or degradation of the polymer over time (e.g., utilizing a biodegradable polymers). In other embodiments, position sensors, as well as other types of sensors, are provided which indicate movement or migration of the polymer in actual use over a period of time.

According to other embodiments, fluid pressure sensors, pulse pressure sensors, liquid (e.g., blood) volume sensors, liquid (e.g., blood) flow sensors, liquid (e.g., blood) chemistry sensors, liquid (e.g., blood) metabolic sensors, contact sensors, and temperature sensors are provided which can monitor the surface environment of the polymer in situ (for example, if the polymer is in the form of a tube or catheter, on both the luminal and adluminal surface). Important changes to the luminal surface such as clotting, obstruction (biliary and urinary “stones”, inflammatory tissue, restenosis), infection (bacteria, fungus, pus, white blood cells, biofilm, etc.), narrowing, increased pressure and changes in flow rates through the tube can be identified in this manner. Also of great value in the continuous monitoring of patient function, status and health are changes in the content (for example: protein, albumin and enzymes; white cells, red cells, hematocrit, cellular casts, bacteria) and/or chemistry (for example: glucose, protein, calcium, nitrite, electrolytes, phosphate, hCG, hemoglobin, ketones, bilirubin, urobiligen, creatinine, urea nitrogen, catecholamines, dopamine, cortisol, specific gravity, osmolality, pH, crystals, liver enzymes, cardiac enzymes, blood lipids, oxygen levels, illicit drug levels, etc.) of the fluids (blood, urine, bile, GI contents, drainage fluids, etc.) flowing through the catheter. In some instances, adluminal surface sensors (fluid pressure sensors, pressure sensors, liquid volume sensors, liquid flow sensors, liquid chemistry sensors, liquid metabolic sensors, contact sensors) are critical for monitoring changes to the outer catheter surface in order to identify abnormalities due to increased pressure (from the presence of a clot, mass, or abscess; leakage; kinking; inadvertent placement or migration into an artery), improper flow (fluids “bypassing” or circumventing the medical polymer (e.g., leakage of a tube), unwanted movement/position/contact (migration into non-target tissues), changes in the chemistry of the fluids around the medical polymer (bleeding, leakage, formation of a fibrin sheath, biofilm or infection) and/or changes in the contact between the medical polymer and the surrounding tissues (correct placement, formation of scar tissue, encapsulation by inflammatory tissue or biofilm, abscess formation).

Within further embodiments, the polymer can contain sensors at specified densities in specific locations. For example, the polymer can have a density of sensors of greater than one, two, three, four, five, six, seven, eight, nine, or ten sensors (e.g., accelerometers (acceleration, tilt, vibration, shock and rotation sensors), pressure sensors, contact sensors, position sensors, chemical sensors, tissue metabolic sensors, mechanical stress sensors and temperature sensors, or any combination of these) per square centimeter of the polymer. Within other embodiments, the medical polymer can have a density of sensors of greater than one, two, three, four, five, six, seven, eight, nine, or ten sensors [e.g., accelerometers (acceleration, tilt, vibration, shock and rotation sensors)], pressure sensors, contact sensors, position sensors, chemical sensors, tissue metabolic sensors, mechanical stress sensors and temperature sensors, or any combination of these) per cubic centimeter of the polymer.

Within certain embodiments of the invention, the polymer is provided with a specific unique identifying number, and within further embodiments, each of the sensors on, in or around the medical polymer each have either a specific unique identification number, or a group identification number (e.g., an identification number that identifies the sensor as accelerometers (acceleration, tilt, vibration, shock and rotation sensors), pressure sensors, contact sensors, position sensors, chemical sensors, tissue metabolic sensors, mechanical stress sensors and temperature sensors). Within yet further embodiments, the specific unique identification number or group identification number is specifically associated with a position on, in or around the medical polymer.

Within other aspects of the invention methods are provided for monitoring an implanted polymer comprising the steps of transmitting a wireless electrical signal from a location outside the body to a location inside the body; receiving the signal at a sensor positioned on, in or around an polymer located inside the body; powering the sensor using the received signal; sensing data at the sensor; and outputting the sensed data from the sensor to a receiving unit located outside of the body.

Within other aspects of the invention methods are provided for imaging a polymer as provided herein, comprising the steps of (a) detecting the location of one or more sensors in a polymer and/or associated medical device; and (b) visually displaying the location of said one or more sensors, such that an image of the polymer is created. Within various embodiments, the step of detecting may be done over time, and the visual display may thus show positional movement over time. Within certain preferred embodiments the image which is displayed is a three-dimensional image.

The imaging techniques provided herein may be utilized for a wide variety of purposes. For example, within one aspect, the imaging techniques may be utilized during a surgical procedure in order to ensure proper placement and working of the polymer. Within other embodiment, the imaging techniques may be utilized post-operatively in order to examine the polymer, and/or to compare operation and/or movement of the polymer over time.

The integrity of the polymer can be wirelessly interrogated and the results reported on a regular basis. This permits the health of the patient to be checked on a regular basis or at any time as desired by the patient and/or physician. Furthermore, the polymer can be wirelessly interrogated when signaled by the patient to do so (via an external signaling/triggering polymer) as part of “event recording”—i.e. when the patient experiences a particular event (e.g. pain, injury, increased or reduced drainage, etc.) she/he signals/triggers the polymer to obtain a simultaneous reading in order to allow the comparison of subjective/symptomatic data to objective/sensor data. Matching event recording data with sensor data can be used as part of an effort to better understand the underlying cause or specific triggers of a patient's particular symptoms. Hence, within various embodiments of the invention methods are provided for detecting and/or recording an event in a subject with one of the polymers provided herein, comprising the interrogating at a desired point in time. Within one aspect of the invention methods are provided for detecting and/or recording an event in a subject with a polymer as provided herein, comprising the step of interrogating at a desired point in time the activity of one or more sensors within the polymer, and recording said activity. Within various embodiments, they may be accomplished by the subject and/or by a health care professional. Within related embodiments, the step of recording may be performed with one or more wired polymers, or, wireless polymers that can be carried, or worn (e.g., a cellphone, watch or wristband, and/or glasses).

Within further embodiments, each of the sensors contains a signal-receiving circuit and a signal output circuit. The signal-receiving circuit receives an interrogation signal that includes both power and data collection request components. Using the power from the interrogation signal, the sensor powers up the parts of the circuitry needed to conduct the sensing, carries out the sensing, and then outputs the data to the interrogation module. The interrogation module acts under control of a control unit which contains the appropriate I/O circuitry, memory, a controller in the form of a microprocessor, and other circuitry in order to drive the interrogation module. Within yet other embodiments the sensor (e.g., accelerometers (acceleration, tilt, vibration, shock and rotation sensors), pressure sensors, contact sensors, position sensors, chemical sensors, tissue metabolic sensors, mechanical stress sensors and temperature sensors) are constructed such that they may readily be incorporated into or otherwise mechanically attached to the polymer (e.g., by way of a an opening or other appendage that provides permanent attachment of the sensor to the polymer) and/or readily incorporated into body of the polymer.

Within yet other aspects of the invention methods polymers having sensors are provided suitable for transmitting a wireless electrical signal from a location outside the body to a location inside the body; receiving the signal at one of the aforementioned sensors positioned on, in or around a polymer located inside the body; powering the sensor using the received signal; sensing data at the sensor; and outputting the sensed data from the sensor to a receiving unit located outside of the body. Within certain embodiments the receiving unit can provide an analysis of the signal provided by the sensor.

The data collected by the sensors can be stored in a memory located within the polymer, or on an associated device (e.g., an associated medical device, or an external device such as a cellphone, watch, wristband, and/or glasses. During a visit to the physician, the data can be downloaded via a wireless sensor, and the doctor is able to obtain data representative of real-time performance of the polymer, and any associated medical device.

The advantages obtained include more accurate monitoring of the polymer and permitting medical reporting of accurate, in situ, data that will contribute to the health of the patient. The details of one or more embodiments are set forth in the description below. Other features, objects and advantages will be apparent from the description, the drawings, and the claims. In addition, the disclosures of all patents and patent applications referenced herein are incorporated by reference in their entirety.

### BRIEF DESCRIPTION OF THE DRAWINGS

FIG. 1 is a representative illustration of various sutures having sensors, including FIG. 1A (a braided suture); FIG. 1B (a chromic suture); FIG. 1C (a polymer-based suture); and FIG. 1D (a metal-based suture).

FIG. 2 is a representative illustration of a barbed suture having sensors.

FIG. 3 is an illustration of representative meshes, including FIG. 3A (a sheet of mesh); FIG. 3B (a blown up illustration of a representative mesh structure having sensors thereon); and FIG. 3C (further magnifications of a representative mesh).

FIG. 4 is an illustration of representative staples having sensors, including FIG. 4A (a staple having various sensors); FIG. 4B (a group of staples having various sensors); and FIG. 4C (an implanted staple having various sensors).

FIG. 5 is an illustration of a representative device for delivery polymers, including for example, FIG. 5A (a syringe having various sensors within the polymer-filled syringe); and FIG. 5B (one of the barrels of the syringe being filled with polymer and various sensors, and the other being filled with a co-polymer).

FIG. 6 illustrates an information and communication technology system embodiment arranged to process sensor data.

FIG. 7 is a block diagram of a sensor, interrogation module, and a control unit according to one embodiment of the invention.

FIG. 8 is a schematic illustration of one or more sensors positioned on a catheter within a subject which is being probed for data and outputting data, according to one embodiment of the invention.

### DETAILED DESCRIPTION OF THE INVENTION

Briefly stated the present invention provides a variety of sensor containing medical polymers. The sensors provided herein can be utilized to monitor the placement, performance, integrity and/or efficaciousness of the polymer and/or other associated medical polymer). Prior to setting forth the invention however, it may be helpful to an understanding thereof to first set forth definitions of certain terms that are used hereinafter.

“Polymer” refers to a macromolecule, typically in excess of 1,000 g/mol, or in excess of 5,000 g/mol molecular weight, or in excess of 10,000 g/mol, which comprises a plurality of repeating units that are present as part of the backbone of the polymer, the plurality typically in excess of 10, or in excess of 20, or in excess of 50.

Polymers may be composed of synthetic materials (e.g., silicone, polyurethane and rubber), composed of non-synthetic components (e.g., harvested grafts for bypass), or some combination of these (e.g., artificial blood vessels having a synthetic polymer scaffold, and naturally occurring cells (e.g., fibroblasts) which produce matrix materials for the vessel (e.g., collagen). Representative examples of polymers include polyester, polyurethanes, silicones, epoxy resin, melamine formaldehyde resin, acetal, polyethyelene terephthalate, polysulphone, polystyrene, polyvinyl chloride, polyamide, polyolefins, polycarbonate, polyethylene, polyamides, polimides, polypropylene, polytetrafluoroethylene, ethylene propylene diene rubber, styrenes (e.g., styrene butadiene rubber), nitriles (e.g., nitrile rubber), hypalon, polysulphide, butyl rubber, silicone rubber, cellulose, chitosan, fibrinogen, collagen, hyaluronic acid, PEEK, PTFE, PLA, PLGA, PCL and PMMA.

The polymer containing sensors of the present invention are preferably suitable for medical applications, and hence are preferably sterile, non-pyrogenic, and/or suitable for use and/or implantation into humans. However, within certain embodiments of the invention the polymer can be made in a non-sterilized environment (or even customized or “printed” for an individual subject), and sterilized at a later point in time.

“Sensor” refers to a polymer that can be utilized to measure one or more different aspects of a body, of a polymer inserted within a body, and/or the integrity, impact, efficaciousness or effect of the polymer inserted within a body. Representative examples of sensors suitable for use within the present invention include, for example, fluid pressure sensors, contact sensors, position sensors, pulse pressure sensors, liquid (e.g., blood) volume sensors, liquid (e.g., blood) flow sensors, chemistry sensors (e.g., for blood and/or other fluids), metabolic sensors (e.g., for blood and/or other fluids), accelerometers, mechanical stress sensors and temperature sensors. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor.

A wide variety of sensors (also referred to as Microelectromechanical Systems or “MEMS”, or Nanoelectromechanical Systems or “NEMS”, and BioMEMS or BioNEMS, see generally https://en.wikipedia.org/wiki/MEMS) can be utilized within the present invention. Representative patents and patent applications include U.S. Pat. Nos. 7,383,071 and 8,634,928, and U.S. Publication Nos. 2010/0285082, and 2013/0215979. Representative publications include “Introduction to BioMEMS” by Albert Foch, CRC Press, 2013; “From MEMS to Bio-MEMS and Bio-NEMS: Manufacturing Techniques and Applications by Marc J. Madou, CRC Press 2011; “Bio-MEMS: Science and Engineering Perspectives, by Simona Badilescu, CRC Press 2011; “Fundamentals of BioMEMS and Medical Micropolymers” by Steven S. Saliterman, SPIE—The International Society of Optical Engineering, 2006; “Bio-MEMS: Technologies and Applications”, edited by Wanjun Wang and Steven A. Soper, CRC Press, 2012; and “Inertial MEMS: Principles and Practice” by Volker Kempe, Cambridge University Press, 2011; Polla, D. L., et al., “Micropolymers in Medicine,” Ann. Rev. Biomed. Eng. 2000, 02:551-576; Yun, K. S., et al., “A Surface-Tension Driven Micropump for Low-voltage and Low-Power Operations,” J. Microelectromechanical Sys., 11:5, October 2002, 454-461; Yeh, R., et al., “Single Mask, Large Force, and Large Displacement Electrostatic Linear Inchworm Motors,” J. Microelectromechanical Sys., 11:4, August 2002, 330-336; and Loh, N. C., et al., “Sub-10 cm³ Interferometric Accelerometer with Nano-g Resolution,” J. Microelectromechanical Sys., 11:3, June 2002, 182-187; all of the above of which are incorporated by reference in their entirety.

Within various embodiments of the invention the sensors described herein may be placed at a variety of locations and in a variety of configurations, on the inside of the polymer, within the body of the polymer, or on the outer surface (or surfaces) of the polymer, between the polymer and any device that might carry or deploy it (e.g., for example, a polymer that is delivered endoscopically). Within certain embodiments the polymer and/or any associated delivery device comprise sensors at a density of greater than 1, 2, 3, 4, 5, 6, 7, 8, 9, 10 or greater than 10 sensors per square centimeter. Within other aspects the polymer and/or associated delivery device comprise sensors at a density of greater than 1, 2, 3, 4, 5, 6, 7, 8, 9, 10 or greater than 10 sensors per cubic centimeter. Within either of these embodiments there can be less than 50, 75, 100, or 100 sensors per square centimeter, or per cubic centimeter. Within various embodiments the at least one or more of the sensors may be placed randomly, or at one or more specific locations within the polymer, and/or associated delivery device.

In various embodiments, the sensors may be placed within specific locations and/or randomly throughout the polymer, and/or associated medical polymer or kit. In addition, the sensors may be placed in specific patterns (e.g., they may be arranged in the pattern of an X, as oval or concentric rings around the polymer and/or associated delivery device.

### Representative Embodiments of Polymers and Medical Uses of Sensor Containing Polymers

In order to further understand the various aspects of the invention provided herein, the following sections are provided below: A. Medical Polymers and their Use; B. Use of Medical Polymers to Deliver Therapeutic Agent(s); C. Use of Polymer having Sensors to Measure Flow and Flow Obstruction; D. Methods for Monitoring Infection in Medical Polymers; E. Further Uses of Sensor-containing Medical Polymers in Healthcare; F. Generation of Power from Medical Polymers; G. Medical Imaging and Self-Diagnosis of Assemblies Comprising Medical Polymers, Predictive Analysis and Predictive Maintenance; H. Methods of Monitoring Assemblies Comprising Medical Polymers; and I. Collection, Transmission, Analysis, and Distribution of Data from Assemblies Comprising Medical Polymers.

### A. Medical Polymers

A1. Medical Polymers Having Sensors

Various polymers may be used in the present invention. Examples include polyester, polyurethane, silicone, epoxy resin, melamine formaldehyde resin, acetal, polyethyelene terephthalate, polysulphone, polystyrene, polyvinyl chloride, polyamide, polycarbonate, polyethylene, polypropylene, polytetrafluoroethylene, ethylene propylene diene rubber, polyurethane rubber, styrene butadiene rubber, nitrile rubber, hypalon, polysulphide, butyl rubber, and silicone rubber. The polymer may be classified by whether it is synthetic or non-synthetic. In addition, or alternatively, it may be classified as being biodegradable or non-biodegradable. In one embodiment, the polymer is a synthetic biodegradable polymer, for example, a co-polymer of lactide and glycolide. In another embodiment, the polymer is a synthetic non-biodegradable polymer, such as polyvinyl chloride. In another embodiment, the polymer is a non-synthetic, i.e., a natural occurring polymer that is biodegradable, such as collagen, fibrinogen, and/or hyaluronic acid. In another aspect, the polymer is a non-synthetic polymer that is non-biodegradable, e.g., cellulose and chitin. Some of these, as well as additional examples, are discussed further below.

The polymer may be a polyester. Polyesters contain repeating ester groups separated by aliphatic or aromatic groups. Polyesters may be formed by reaction between a di-acid (e.g., adipic acid, phthalic acid) and a di-alcohol (e.g., ethylene glycol, butylene glycol), or reactive equivalents thereof. The polyester may be biodegradable, such as polylactic acid (PLA), poly (lactic-co-glycolic) acid (PLGA), and polycaprolactone (PCL).

The polymer may be a polyether, optionally including other repeating units. For example, the polymer may be a polyetherimide, having both repeating ether and imide groups. As another example, the polymer may be a polyethersulfone, with repeating ether and sulfone groups.

The polymer may be characterized in terms of its thermal properties. For example, in one embodiment the polymer is a thermoplastic. A thermoplastic becomes plastic (i.e., fluid) upon heating and hardens upon cooling and is able to repeat this phase change multiple times in response to changes in temperature. Examples of thermoplastics include PET, polysulphone, polystyrene, UPVC, polyamides, polycarbonates, polyethylene, polypropylene and PTFE. In another embodiment the polymer is a thermoset. A thermoset is does not become fluid upon heating, but instead retains it hardened form even at elevated temperature. Examples of thermosets include epoxy and phenolics.

The polymer may be a phenolic. Many phenolic polymers are thermoset. Phenolic resins are typically formed between a phenol and formaldehyde, and is sometimes referred to a phenol formaldehyde resin. Novolacs are phenolics made with a formaldehyde to phenol molar ratio of less than one, while resoles are phenolics made with a formaldehyde to phenol ratio of greater than one (usually around 1.5).

The polymer may be an epoxy. Many epoxy polymers are thermoset. Hardened epoxy resins are formed between a polyepoxide compound (often a di-epoxide) and a curing agent such as a poly-hydroxyl or poly-amine. A common epoxy resin is the reaction product between epichlorohydrin and bisphenol A to form diglycidyl ethers of bisphenol A. A common curing agent is triethylenetetramine. Epoxy resins may also be thermally cured. Epoxy resins are tough and resistant to many environments, making them useful components of many medical polymers.

The polymer may be a polyolefin. Many polyolefin polymers are thermoplastic. Exemplary polyolefins are polyethylene (PE) and polypropylene (PP). Polyolefins are commercially available in a wide range of molecular weights, and different molecular weights have different properties and different applications. For example, ultra-high molecular weight PE may be used to prepare load bearing materials in total joint replacements.

The polymer may be acrylonitrile butadiene styrene (ABS), which is typically a thermoplastic. As its name suggests, ABS is formed by copolymerization of the monomers acrylonitrile, butadiene and styrene. ABS may be viewed as a styrene-acrylonitrile copolymer modified by butadiene rubber. ABS combines the resilience of polybutadiene with the hardness and rigidity of polyacrylonitrile and polystyrene. The properties of the ABS polymer depend to a large extent on the relative amount of each of the monomers used in its preparation. Acrylonitrile tends to impart chemical resistance, heat stability, increased tensile strength, and aging resistance. Styrene tends to impart gloss and rigidity, and also help aid is processing the plastic. Butadiene imparts toughness, impact strength, good low temperature properties.

The polymer may be an ethylene vinyl alcohol (EVA, or EVAL or EVOH) copolymer which is formed by copolymerization of ethylene and vinyl acetate, whereupon the acetate groups are hydrolyzed to hydroxyl (alcohol) groups. EVOH is biocompatible and biodegradable. EVOH is recognized as having excellent bather properties to oxygen, and accordingly is often used as a coating to provide this desirable function.

The polymer may be a fluoroplastic. As used herein, a fluoroplastic refers to a polymer that is a thermoplastic and which contains carbon-fluorine bonds. Examples are poly(tetrafluoroethylene), also known as PTFE.

The polymer may be polyvinyl chloride (PVC). PVC comes in two basic grades: flexible and rigid. The flexible form is typically prepared by incorporation of various additives into the PVC, where exemplary additives are plasticizers (e.g., phthalates) and stabilizers. Flexible PVC is used in many medical applications due to its biocompatibility, transparency, softness, light weight, high tear strength, kink resistance, and suitability for sterilization. PVC may be chlorinated to increase its chlorine content, thereby creating CPVC.

The polymer may be polysulfone (PS). For example, the polymer may be a polyphenylsulfone. Westlake Plastics (Lenni, Pa.) markets medical grade Radel R5500 polyphenylsulfone resin. This polymer provides hydrolytic stability, toughness, and good impact strength over a wide temperature range. Recommended sterilization techniques for Radel R5500 include EtO gas, radiation, steam autoclaving, dry heat and cold sterilization.

The polymer may be polyether ether ketone (PEEK). An exemplary PEEK polymer is formed by reaction of 4,4′-difluorobenzophenone with the disodium salt of hydroquinone. PEEK is a semicrystalline, high-temperature (up to 500° F.) engineering thermoplastic that is useful in applications where thermal, chemical, and combustion properties are important to performance. PEEK also resists radiation and a wide range of solvents including water. With its resistance to hydrolysis, PEEK can withstand boiling water and superheated steam used with autoclave and sterilization equipment at temperatures higher than 482° F., thus making it useful in the manufacture of many medical parts.

The polymer may be polycarbonate (PC). For example, Westlake Plastics (Lenni, Pa.) markets medical grade Zelux GS polycarbonate which may be sterilized by EtO gas and limited autoclaving sterilization.

The polymer may be a polyimide, such as a polyetherimide. For example, Westlake Plastics (Lenni, Pa.) markets medical grade Tempalux polyetherimide. This polymer maintains its size and shape over a broad temperature range as well as tolerates a high amount of stress over extended periods of time. Recommended sterilization techniques for Tempalux include EtO gas, radiation, steam autoclaving, dry heat and cold sterilization.

The polymer may comprise repeating oxymethylene units. For example, the polymer may be a homopolymer of oxymethylene units, which is known polyoxymethylene (POM) or acetal or polyacetal. The term POM will be used to refer to homopolymers prepared from formaldehyde or equivalent, which may have various endgroups to enhance the stability of the homopolymer. When a high molecular weight version of the homopolymer is reacted with acetic anhydride, the resulting product is hard, rigid and has high strength. A version is sold by du Pont (Wilmington Del.) as their Delrin polymer and advertised for use in medical products. The polymer may be a copolymer including repeating oxymethylene units. For example, formaldehyde may be converted to 1,3,5-trioxane, which in turn is reacted with a suitable co-monomer such as ethylene oxide or dioxolane. Hostaform from Ticona (now Celanese, Irving, Tex.) and Ultraform from BASF (Florham Park, N.J.) are two examples of commercially available oxymethylene copolymers. Polyplastics (Taipei, Taiwan) manufactures DURACON POM, which may be used in medical products. TECAFORM MT is a POM manufactured by Ensinger Inc. (Washington, Pa.) which is particularly suited for use as sizing trials in knee, hip and shoulder replacement procedures.

The polymer may be characterized in terms of its viscoelastic properties. For example, in one embodiment the polymer is elastic, in which case the polymer may be referred to as an elastomer. At ambient temperatures, elastomers are relatively soft and deformable, i.e., they may be stretched and will return back to its original shape after the stretching force is removed. One type of elastomer is a rubber, where a rubber is typically formed by a process that includes vulcanization. Alternatively, the polymer may be rigid and non-deformable.

The polymer may be a polyurethane. Polyurethanes are formed when a polyol (i.e., a polyhydroxylated compound) reacts with a diisocyanate or a polymeric isocyanate when there are suitable catalysts and additives present. The polyurethane may be a thermoset, particularly when crosslinking reactants are used in its preparation. Alternatively, the polyurethane polymer may be an elastomer. For example, Bayer (Leverkusen, Germany) markets Vulkollan® polymer which is produced by reacting polyesterpolyols, Desmodur® 15 (one or both of MDI (diphenylmethane diisocyanate) and TDI (toluylene diisocyanate) and glycols at temperatures exceeding 100° C. in a multistage process. Vulkollan® polymer may be formed into parts and is particularly well-suited when high mechanical load bearing and high dynamic load bearing capacity is needed. Another suitable polyurethane elastomer, also from Bayer, is Baytec® Spray, a material consisting of two liquid, polyurethane-based components. Baytec® Spray can be used to provide an elastomeric coating on the surface of a polymer.

The polymer may be a natural polymer or a synthetic polymer. A natural polymer is found in nature, where rubber is an example of a natural polymer. A synthetic polymer is not found in nature but is instead made through human-controlled chemical reactions. Polyurethanes are exemplary synthetic polymers. Carbohydrates (e.g., cellulose, hyaluronic acid) and poly(amino acid) (e.g., protein, collagen) are examples of natural polymers. Cellulose finds use in, e.g., the manufacture of dialysis membranes. Chitin is a natural polymer, however the synthetic deacylation of chitin produces the synthetic polymer chitosan. Hyaluronic acid is a natural polymer that finds use in the treatment of osteoarthritis and other joint disorders.

The polymer may be a synthetic elastomer, also known as a synthetic rubber. There are several well-known synthetic elastomers, which are named from the monomer(s) from which they are produced. Those elastomers include cis-polybutadiene (butadiene rubber, BR), styrene-butadiene rubber (SBR), ethylene-propylene monomer (EPM), acrylonitrile-butadiene copolymer (nitrile rubber), isobutylene-isoprene copolymer (butyl rubber), ethylene-propylene-diene monomer (EPDM, where the diene may be, e.g., butadiene), and polychloroprene (neoprene). In large part these synthetic rubbers consist of two or more different monomer units, e.g., styrene and butadiene, arranged randomly along the molecular chain. EPM and nitrile rubber also consist of a random arrangement of two monomers—in this case, ethylene and propylene (which form EPM) and butadiene and acrylonitrile (which form nitrile rubber). Another suitable rubber is silicon rubber, which finds widespread use in catheters and other types of medical tubing. Silicon rubber may be prepared by curing a liquid precursor, e.g., with a platinum catalyst, usually at elevated temperature. The glass transition temperatures of all these polymers are quite low, well below room temperature, so that all of them are soft, highly flexible, and elastic. The present disclosure provides that any one or more of the named synthetic rubbers may be used in the compositions and methods as identified herein.

Instead of an organic polymer, the polymer or coating may be formed in whole or in part from a ceramic biomaterial, sometimes referred to as a bioceramic. An example of a ceramic biomaterial is hydroxyapatite, which may be combined with a binder to create a solid mass or a coating. Suitable binders include collagen, gelatin, and polyvinylalcohol. A sol-gel process may be used to prepare the final product. Other examples of bioceramics include alumina (Al₂O₃) and zirconia (ZrO₂), tricalcium phosphate (Ca₃(PO₄)₂), and bioglass (Na₂OCaOP₂O₃—SiO). The bioceramic may be biodegradable (e.g., tricalcium phosphate) or biostable (e.g., alumina). The bioceramics alumina and zirconia are used in orthopedics to produce, for example, femoral heads, artificial knees, bone screws and bone plates, and in dental applications are used to produce crowns and bridges.

The medical polymer may be multi-component. For example, it may be a blend of two or more polymers. As another example, it may be a composite of organic and inorganic materials. For example, the medical polymer may be a blend of polyester and a mineral component, or a blend of silicone and a mineral component.

A2. Manufacture of Medical Polymers

A polymer may be fabricated into a desired shape for a medical polymer by various methods including extrusion, molding (e.g., injection molding, compression molding) thermoforming, electrospinning, and cutting (e.g., stamping, die cutting). During the fabrication process, a sensor may be incorporated into the polymer.

For example, the polymer may be fabricated by a thermoforming technique, including vacuum, pressure and mechanical types of thermoforming. In general, thermoforming refers to a process of converting an initially flat thermoplastic sheet into a desired three-dimensional shape, where the process includes at least two stages: softening the sheet by heating, followed by forming it in a mold cavity. In vacuum thermoforming, the heated thermoplastic sheet is held in the cavity by means of vacuum produced between the sheet and the surface of the mold cavity space. In pressure thermoforming, gas pressure is applied against the heated sheet in the direction of the mold cavity, thereby forcing the sheet against the contours of the cavity. In mechanical thermoforming, a solid object is pushed against the sheet so that the sheet is forced against the contour of the mold. Upon cooling, the thermoplastic sheet adopts the shape of the mold. A sensor may be placed in the heated sheet before or during the forming process, so that upon cooling, the sheet adopts a desired shape and the sensor is embedded in whole or part in the thermoplastic sheet.

As another example, the polymer may be fabricated by a molding process, whereby solid or molten polymer or pre-polymer is placed within a mold. Upon cooling, the polymer will adopt the configuration of the mold. Various types of molding process that may be used. For example, compression molding squeezes a pre-polymer into a pre-heated mold and then applies heat and pressure to the pre-polymer, causing the pre-polymer to cure into the shape of the mold. This process may be used for both thermoplastic and thermosetting polymer. In blow molding, a heated hollow thermoplastic tube is inflated within a closed mold until it adopts the shape of the mold. Upon cooling, the newly shaped tube will retain the shape of the mold.

Electrospinning is particularly suited for preparing polymeric fibers, and represents another example of fabricating a polymer. For example, it can be used to form nanofibers from various organic polymers. See, e.g., Doshi, J. and Reneker, D. H., Journal of Electrostatics 35(2-3):151-160, 1995. Fibers formed from electrospinning may be made into various shapes, including matrices formed from woven and non-woven fibers. Sensors may be embedded within the matrix formed from the electrospun fibers.

As yet another example, the medical article may be formed by any of weaving, plying, braiding, knitting, and stitching of polymeric fibers. These processes may be used to form various shapes, including a sheet (as found, e.g., in a mesh), filament (as found, e.g., in a suture), and a tube (as found, e.g., in a graft). See, e.g., U.S. Pat. No. 5,378,469 directed to high strength collagen threads, which are optionally crosslinked, where the threads may be used to form braided constructs, plied into yarn, and knitted to provide an implant. A sensor as described herein can be incorporated in, or associated with, the braided, knitted, or woven materials.

The medical polymer may be sterilized by techniques known in the art. For example, the medical polymer may be exposed to ionizing radiation, such as gamma radiation and electron beam radiation. While ionizing radiation may sterilize the medical polymer, it can also cause some breakdown of the polymer's basic structure. To combat this problem, stabilizers may be added to the polymer, where examples include antioxidants such as phenolics that react with free radicals, and organo-phosphorous compounds which react with peroxide and hydroperoxides generated by the reaction of oxygen with reactive sites generated by the ionizing radiation. Another sterilization technique is to expose the medical polymer to ethyelene oxide. An advantage of ethylene oxide sterilization is that it is not harmful to the structure of the polymer, and accordingly is a suitable sterilization technique when a medical polymer must be repeated sterilized. Another sterilization technique is to expose the medical polymer to high temperature, optionally in the presence of steam, e.g., in an autoclave.

Within various embodiments of the invention, methods are also provided for manufacturing a medical polymer having one of the sensors provided herein. For example, within one embodiment of the invention a medical polymer is constructed such that one or more sensors provided herein are placed directly into, onto, or within the medical polymer at the time of manufacture, and subsequently sterilized in a manner suitable for use in subjects.

Within other embodiments, scaffolds can be prepared from medical polymers (see, e.g., U.S. Pat. No. 8,562,671, and WO 2013/142879 which are incorporated by reference in their entirety). Briefly, scaffolds composed of one or more medical polymers can be prepared in order to mimic the shape of a biological structure (e.g., vessel), or a portion thereof. Sensors can be placed into the structure before, during, or subsequent to manufacture of the valve (e.g., in the case or electro-spinning or molding of polymer fibers, or in the case of 3D printing as described in more detail below). Within certain preferred embodiments the scaffold can be seed with stem cells suitable for growth of tissue on the artificial medical polymer (see, e.g., WO 1999/003973 and U.S. Pat. No. 8,852,571, which are incorporated by reference in their entirety).

Within further embodiments, the present disclosure provides a method of making a medical polymer by 3D printing, additive manufacturing, or a similar process whereby the medical polymer is formed from powder or filament that is converted to a fluid form such subsequently solidifies as the desired shape. For convenience, such processes will be referred to herein as printing processes or 3D printing processes. The present disclosure provide a method of making a medical polymer by a printing process, where that medical polymer includes a sensor, circuit or other feature as disclosed herein (collectively sensor or sensors). The sensor may be separately produced and then incorporated into the medical polymer during the printing process. For example, a sensor may be placed into a desired position and the printing process is carried out around the sensor so that the sensor becomes embedded in the printed medical polymer. Alternatively, the printing process may be started and then at appropriate times, the process is paused to allow a sensor to be placed adjacent to the partially completed medical polymer. The printing process is then re-started and construction of the medical polymer is completed. The software that directs the printing process may be programmed to pause at appropriate predetermined times to allow a sensor to be added to the partially printed medical polymer.

In addition, or alternatively, the sensor itself, or a portion thereof may be printed by the 3D printing process. Likewise, electronic connectively to, or from, or between, sensors may be printed by the 3D printing process. For example, conductive silver inks may be deposited during the printing process to thereby allow conductivity to, or from, or between sensors of a medical polymer. See, e.g., PCT publication nos. WO 2014/085170; WO 2013/096664; WO 2011/126706; and WO 2010/0040034 and US publication nos. US 2011/0059234; and US 2010/0037731. Thus, in various embodiments, the present disclosure provides medical polymers wherein the sensor is printed onto a substrate, or a substrate is printed and a sensor is embedded or otherwise incorporated into or onto the substrate, or both the substrate and the sensor are printed by a 3D printing technique.

3D printing may be performed using various printing materials, typically delivered to the 3D printer in the form of a filament. Two common printing materials are polylactic acid (PLA) and acrylonitrile-butadiene-styrene (ABS), each being an example of a thermoplastic polymer. When strength and/or temperature resistance is particularly desirable, then polycarbonate (PC) may be used as the printing material. Other polymers may also be used. See, e.g., PCT publication nos. WO 2014/081594 for a disclosure of polyamide printing material. When metal parts are desired, a filament may be prepared from metal or metal alloy, along with a carrier material which ultimately will be washed or burned or otherwise removed from the part after the metal or metal alloy has been delivered.

When the medical polymer is of a particularly intricate shape, it may be printed with two materials. The first material is cured (using, e.g., actinic radiation) as it is deposited, while the second material is uncured and can be washed away after the medical polymer has been finally printed. In this way, significant hollow spaces may be incorporated into the medical polymer.

Additive manufacturing is a term sometimes used to encompass printing techniques wherein metal or metal allow is the material from which the desired part is made. Such additive manufacturing processes utilizes lasers and build an object by adding ultrathin layers of materials one by one. For example, a computer-controlled laser may be used to direct pinpoint beams of energy onto a bed of cobalt-chromium alloy powder, thereby melting the alloy in the desired area and creating a 10-30-micron thick layer. Adjacent layers are sequentially and repetitively produced to create the desired sized item. As needed, a sensor may be embedded into the alloy powder bed, and the laser melts the powder around the sensor so as to incorporate the sensor into the final product. Other alloys, including titanium, aluminum, and nickel-chromium alloys, may also be used in the additive manufacturing process. See, e.g., PCT publication nos. WO 2014/083277; WO 2014/074947; WO 2014/071968; and WO 2014/071135; as well as US publication nos. US 2014/077421; and US 2014/053956.

Accordingly, in one embodiment the present disclosure provides a method of fabricating a sensor-containing medical polymer, the method comprising forming at least one of a sensor and a support for the sensor using a 3D printing technique. Optionally, the 3D printing technique may be an additive manufacturing technique. In a related embodiment, the present disclosure provides a medical polymer that is produced by a process comprising a 3D printing process, such as an additive manufacturing process, where the medical polymer includes a sensor.

Disclosure of 3D printing processes and/or additive manufacturing is found in, for example PCT publication nos. WO 2014/020085; WO 2014/018100; WO 2013/179017; WO 2013/163585; WO 2013/155500; WO 2013/152805; WO 2013/152751; WO 2013/140147 and US publication nos. 2014/048970; 2014/034626; US 2013/337256; 2013/329258; US 2013/270750.

A3. Use of Medical Polymers in Medical Polymers and Implants

Polymers containing sensors can be utilized in a wide variety of medical devices and implants, including for example, hip and knee prosthesis, tubes (e.g., grafts and catheters), implants (e.g., breast implants), spinal implants, orthopedic and general surgery implants, and cardiovascular implants (e.g., stents, stent grafts, and heart valves). Representative examples of such implants are discussed in more detail in International Patent Application No. PCT/US2013/077356; International Patent Application No. PCT/US2014/028323; International Patent Application No. PCT/US2014/028381; International Patent Application No. PCT/US2014/043736; U.S. Provisional Patent Application Entitled ‘Devices, Systems and Methods for Using and Monitoring Catheters’, filed Jun. 25, 2014, Attorney Docket No. CANA.405P1; U.S. Patent Provisional Application Entitled ‘Devices, Systems and Methods for Using and Monitoring Implants, filed Jun. 25, 2014, Attorney Docket No. CANA.406P1; U.S. Patent Provisional Application Entitled ‘Devices, Systems and Methods for Using and Monitoring Spinal Implants’, filed Jun. 25, 2014, Attorney Docket No. CANA.407P1; U.S. Patent Provisional Application Entitled ‘Devices, Systems and Methods for Using and Monitoring Orthopedic Hardware’, filed Jun. 25, 2014, Attorney Docket No. CANA.408P1; U.S. Patent Provisional Application Entitled ‘Devices, Systems and Methods for Monitoring Heart Valves’, filed Jun. 25, 2014, Attorney Docket No. CANA.410P1; all of the aforementioned patent applications incorporated herein by reference in their entireties for all purposes.

Some additional discussion of medical polymers and devices that can be used in the present invention is as follows:

A3.1 Glues, Adhesives and Cements

The medical polymer may be useful to hold tissue together, or to hold tissue together with a medical implant, such as a glue or adhesive, where the tissue includes soft tissue or bone. When used in bone, the medical polymer is frequently referred to as a bone cement, where bone cement is also used to fill in cavities of bone. For example, the polymer may be the reaction product of two synthetic polyethylene glycols which have reactive endgroups such that upon forming a mixture of the two components, the two materials react with one another and form a crosslinked film. A version of this material is commercially available as COSEAL (Baxter Healthcare, Fremont, Calif., USA). See, e.g., Cannata, A., et al., Ann. Thorac. Surg. 2013, 95:1818-1826. COSEAL may be spayed over a large area, and to varying depths, to provide a glue or adhesive layer on living tissue. A modified chitosan-dextran gel as prepared by the process described in Liu G., et al. Macromolecular Symposia 2009 279:151. See, e.g., Lauder, C. I. W., et al. Journal of Surgical Research 2012 176:448-454. This material may be applied to soft tissue and will function to hold the tissue together. A sprayable material that functions primarily as a barrier but also has some adhesive properties is marketed by Covidien and known as SprayShield. SprayShield is a synthetic two-component product that forms a gel when applied to an organ.

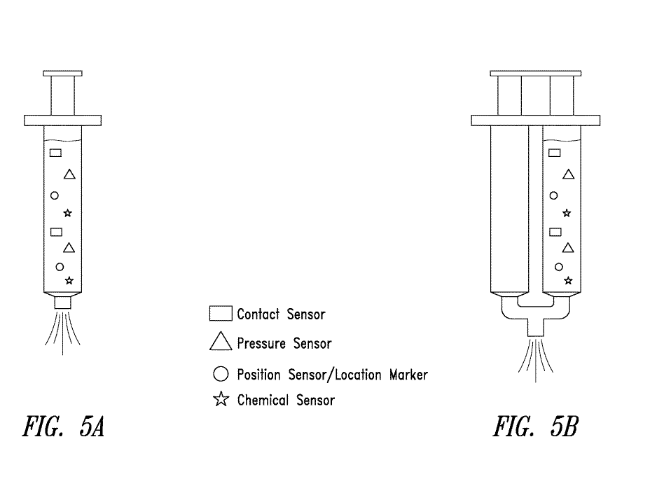

As an example, FIG. 5 illustrates one embodiment of a representative device for delivering a sensor containing polymer. FIG. 5A depicts a syringe containing a flowable polymer, and further comprising a variety of different sensors suitable for the desired indication. FIG. 5B depicts a dual barrel syringe (e.g., containing COSEAL, or another set of two polymers that are designed to be admixed prior or during administration). In this representative embodiment sensors are deployed from only one side of the polymer containing syringe, although, of course they could equally be deployed from both sides. Within various embodiments one or more sensors (e.g., fluid pressure sensors, contact sensors, position sensors, pulse pressure sensors, liquid (e.g., blood) volume sensors, liquid (e.g., blood) flow sensors, chemistry sensors (e.g., for blood and/or other fluids), metabolic sensors (e.g., for blood and/or other fluids), accelerometers, mechanical stress sensors and temperature sensors) can be incorporated into one or more polymers.

A3.2 Medical Polymers—Meshes and Films

Various medical polymers are used to form implantable films of meshes. For example, the biodegradable copolymer of hydroxybutyrate and hydroxyvalerate known as (PHBV) is available from Metabolix, Inc. (Cambridge, N.J., USA) and can function as a barrier film. Oxidized regenerated cellulose is commercially available as Interceed (Johnson & Johnson, Canada), which is a knitted fabric that converts to a gel within 8 hours and is completely cleared from the body within 28 days. See, e.g., Larsson B., J. Reprod. Med. 1996, 41:27-34 and ten Broek R. P. G., et al., The Lancet 2014 383:48-59. Collagen foil in combination with polypropylene mesh is commercially available as TissueFoil E from Baxter (Germany). See, e.g., Schonleben, F., Int. J. Colorectal Dis. 2006, 21(8):840-6. INTERCOAT, also known as OXIPLEX AP, made by Johnson & Johnson and licensed from Fziomed, may be used as an implantable film. PREVADH, made by Sofradim-Covidien in France is a collagen film and fleece composite that may be used as an implantable filem. W. L. Gore manufactures and sells non-absorbable adhesion bather films using expanded polytetrafluoroethylene film, sometimes referred to as GoreTex Surgical Membrane or as Preclude. Each of these films may be used as a medical polymer according to the present invention.

Meshes are available from various vendors. For example, Ethicon markets a synthetic mesh, PROLENE mesh, made from polypropylene. Biological meshes are also known and may be used in the present invention. Examples are meshes formed from human or animal dermis or porcine small intestinal submucosa. See, e.g., Nguyen et al., JAMA Surg., epub Feb. 19, 2014 and Carbonell et al., J. Am. Coll. Surg., 217(6):991-998, 2013.

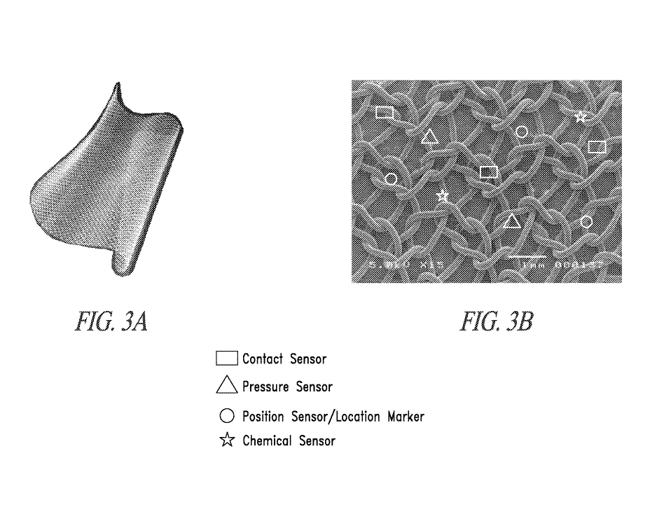

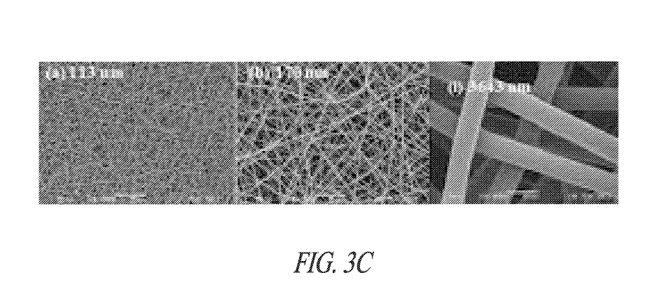

Within one embodiment of the invention one or more sensors can be incorporated into a mesh. For example, as shown in FIGS. 3A to 3C, a variety of sensors can be incorporated into a mesh. Representative examples of sensors include fluid pressure sensors, contact sensors, position sensors, pulse pressure sensors, liquid (e.g., blood) volume sensors, liquid (e.g., blood) flow sensors, chemistry sensors (e.g., for blood and/or other fluids), metabolic sensors (e.g., for blood and/or other fluids), accelerometers, mechanical stress sensors and temperature sensors. Sensors within a mesh or film can be utilized to determine contact between various organs or anatomical structures (e.g. utilizing contact sensors and/or pressure sensors); the presence of or development of an infection (e.g., utilizing temperature and/or metabolic sensors), to determine degradation, wear, movement and/or fracture (e.g., utilizing contact sensors, pressure sensors, and/or location sensors).

A3.3 Medical Polymers—Suture and Staples

The medical polymer may be formed into a device for securing or fastening tissue, such as a staple or a suture. See, e.g., U.S. Pat. Nos. 8,506,591 and 8,721,681 as well as U.S. Publication Nos. 2001/0027322, 2006/0253131, 2011/0093010, 2013/0165971, and 2014/0130326 for exemplary suitable staples and discussion of insertion devices. The medical polymer may be formed into a suture, e.g., PROLENE polypropylene suture by Ethicon (New Jersey), or DEKLENE polypropylene suture sold by Teleflex Medical (North Carolina). See also, e.g., U.S. Pat. Nos. 6,908,466; 4,750,492; 4,662,068 for medical fasteners prepared in whole or part from polymer.

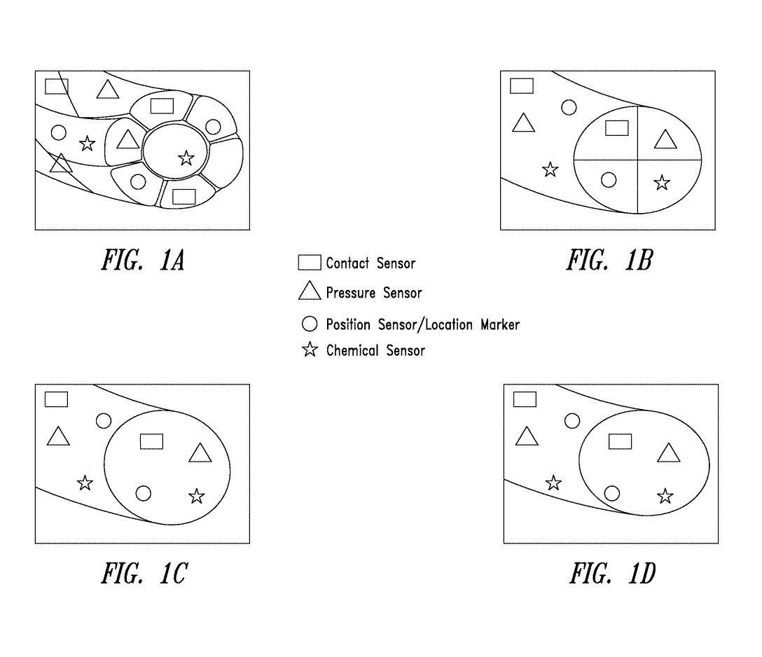

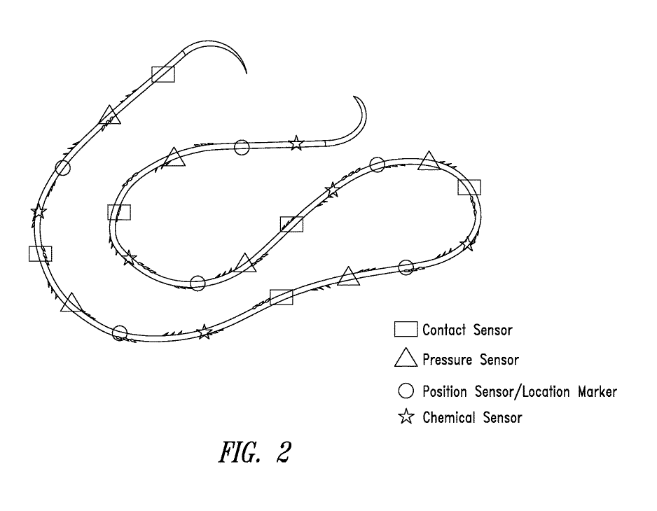

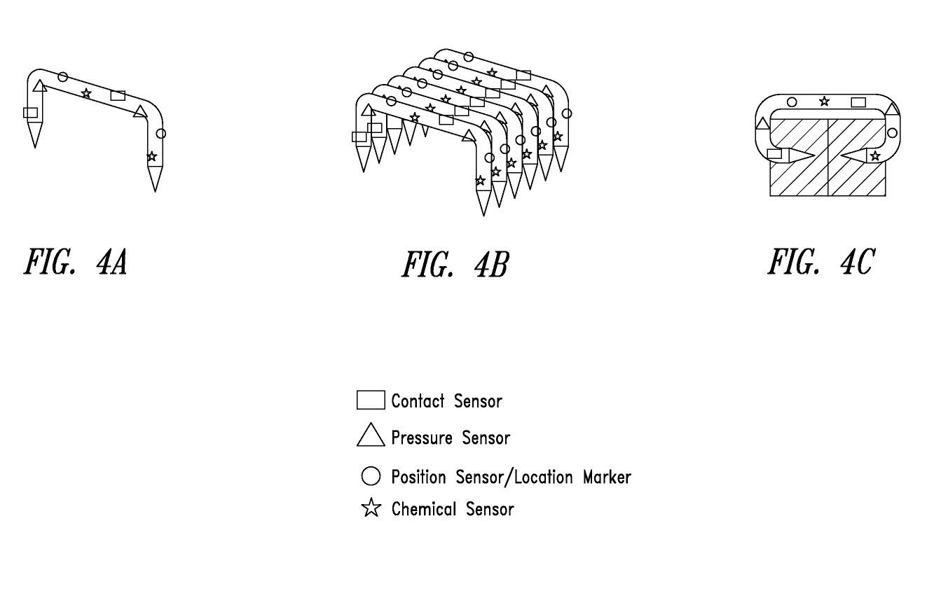

Within one embodiment of the invention one or more sensors can be incorporated into a fixation device such as a suture or staple. For example, as shown in FIGS. 1A to 1D and FIG. 2, a variety of sensors can be incorporated into a suture. Similarly, as shown in FIGS. 4A, 4B and 4C, a variety of sensors can be incorporated into a staple. Representative examples of sensors include fluid pressure sensors, contact sensors, position sensors, pulse pressure sensors, liquid (e.g., blood) volume sensors, liquid (e.g., blood) flow sensors, chemistry sensors (e.g., for blood and/or other fluids), metabolic sensors (e.g., for blood and/or other fluids), accelerometers, mechanical stress sensors and temperature sensors. Sensors within a suture or staple can be utilized to determine contact with various organs or anatomical structures (e.g. utilizing contact sensors and/or pressure sensors); the presence of or development of an infection (e.g., utilizing temperature and/or metabolic sensors), to determine degradation, wear, movement and/or fracture (e.g., utilizing contact sensors, pressure sensors, and/or location sensors).

A4. Medical Polymers—Incorporation of Sensors

As noted above, any of the aforementioned polymers (including for example, polymers such as polyester, polyurethane, silicone, epoxy resin, melamine formaldehyde resin, acetal, polyethyelene terephthalate, polysulphone, polystyrene, polyvinyl chloride, polyamide, polycarbonate, polyethylene, polypropylene, polytetrafluoroethylene, ethylene propylene diene rubber, polyurethanes, styrenes (e.g., styrene butadiene rubber), nitriles (e.g., nitrile rubber), hypalon, polysulphide, butyl rubber, various silicones (e.g. silicone rubber), cellulose, chitosan, fibrinogen, collagen, and hyaluronic acid. Within various embodiments one or more sensors (e.g., fluid pressure sensors, contact sensors, position sensors, pulse pressure sensors, liquid (e.g., blood) volume sensors, liquid (e.g., blood) flow sensors, chemistry sensors (e.g., for blood and/or other fluids), metabolic sensors (e.g., for blood and/or other fluids), accelerometers, mechanical stress sensors and temperature sensors) can be incorporated into one or more polymers. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor.

Within various embodiments of the invention, pressure sensors can be incorporated into a polymer (e.g., for a catheter, on the outer (adluminal) walls, or, within the body of the catheter itself). Such sensors are able to measure pressure in or against the vessel wall. Increased pressures can be suggestive of stenosis, thrombosis or kinking upstream from an obstructing event, whereas decreased pressures would be seen downstream from an obstruction. Having the ability to measure arterial pressure throughout the catheter allows for hemodynamic monitoring of the catheter, and the capability of detection events prior to a complication developing.

Within yet other embodiments contact sensors can be placed on and throughout a polymer (e.g., a catheter) in order to measure contact (integrity of the seal) between the bypass catheter and the vessel to which it is attached. For example, chemical sensors can also be place on and throughout the polymer in order to measure a wide variety of metabolic parameters, including for example: Blood Oxygen content; Blood CO₂ content; Blood pH; Blood cholesterol; Blood lipids (HDL, LDL); Blood Glucose; Cardiac enzymes; Hepatic Enzymes; and Kidney Function (BUN, Creatinine, etc.).

Within other embodiments position sensors can be placed throughout a polymer (e.g., for a catheter on both the luminal and adluminal surfaces, and within the catheter material itself) in order to allow imaging of the polymer, and detection of changes and/or movement over time.

Taken collectively, a wide variety of sensors as described herein can be utilized to detect, measure and assess a number of factors relevant to, for example, cardiac function. For example, blood flow rate detectors, blood pressure detectors, and blood volume detectors (e.g., to measure blood volume over a unit of time) can be placed within (on the luminal side), and on other parts of a polymer (e.g. a catheter) in order to measure systolic and diastolic pressure, cardiac output, ejection fraction, cardiac index and systemic vascular resistance.

Within particularly preferred embodiments such sensors can also be utilized to detect cardiac output (which is a key clinical measurement that must be monitored in compromised patients). For example, high-fidelity pressure transducers can be located on, in, or within a catheter in order to measure the timing and pressure of pulsations. Such measurements can be utilized to assess stroke volume and systemic vascular resistance, and also provide continuous cardiac output monitoring and heart rate monitoring.

Within yet other embodiments chemical and temperature sensors can be utilized to monitor changes in temperature, and/or the presence of an infection or a developing infection.

In summary, a wide variety of sensors may be placed on and/or within the polymers described herein, in order to provide “real time” information and feedback to a health care provider (or a surgeon during a surgical procedure), to detect proper placement, anatomy, alignment, forces exerted on surrounding tissues, and to detect the strain encountered in a surgical procedure. For example, the polymers described herein (e.g. polyester, polyurethanes, silicones, epoxy resin, melamine formaldehyde resin, acetal, polyethyelene terephthalate, polysulphone, polystyrene, polyvinyl chloride, polyamide, polyolefins, polycarbonate, polyethylene, polyamides, polimides, polypropylene, polytetrafluoroethylene, ethylene propylene diene rubber, styrenes (e.g., styrene butadiene rubber), nitriles (e.g., nitrile rubber), hypalon, polysulphide, butyl rubber, silicone rubber, cellulose, chitosan, fibrinogen, collagen, hyaluronic acid, PEEK, PTFE, PLA, PLGA, PCL and PMMA.) provided herein can have one or more contact sensors, strain gauge sensors, pressure sensors, fluid pressure sensors, position sensors, accelerometers, shock sensors, rotation sensors, vibration sensors, tilt sensors, pressure sensors, tissue chemistry sensors, tissue metabolic sensors, mechanical stress sensors and temperature sensors. Sensors can be placed at a density of greater than 1, 2, 3, 4, 5, 6, 7, 8, 9, 10 or greater than 10 sensors per square centimeter or at a density of greater than 1, 2, 3, 4, 5, 6, 7, 8, 9, 10 or greater than 10 sensors per cubic centimeter. Within either of these embodiments there can be less than 50, 75, 100, or 100 sensors per square centimeter, or per cubic centimeter.

The above sensors may be continuously monitored in order to provide a ‘real-world’ activity, healing, and changes in function over time, to evaluate patient activity, and to better understand the conditions which catheters (e.g., hemodialysis catheters) are exposed to in the real world.

### B. Use of Medical Polymers to Deliver Therapeutic Agent(s)

As noted above, the present invention also provides drug-eluting polymers which comprise one or more sensors, and which can be utilized to release a therapeutic agent (e.g., a drug) to a desired location within the body (e.g., a body lumen). For example, anti-restenotic drugs (e.g., paclitaxel, sirolimus, or an analog or derivative of these), can be administered to an atherosclerotic lesion utilizing a drug-eluting polymer (e.g., a balloon catheter or a drug-coated balloon catheter as described in U.S. Pat. No. 7,491,188, U.S. Patent Application Nos. 2006/0079836, US 2009/0254063, US 2010/0023108, and US 2010/0042121). Within preferred embodiments one or more sensors (e.g., pressure sensors, contact sensors, and/or position sensors) can be utilized to determine appropriate placement of the desired drug, as well as the quantity of drug that is released at the desired site.

Within other embodiments of the invention a wide variety of additional therapeutic agents may be delivered (e.g., to prevent or treat an infection or to treat another disease state), including for example: Anthracyclines (e.g., gentamycin, tobramycin, doxorubicin and mitoxantrone); Fluoropyrimidines (e.g., 5-FU); Folic acid antagonists (e.g., methotrexate); Podophylotoxins (e.g., etoposide); Camptothecins; Hydroxyureas, and Platinum complexes (e.g., cisplatin) (see e.g., U.S. Pat. No. 8,372,420 which is incorporated by reference in its entirety. Other therapeutic agents include beta-lactam antibiotics (e.g., the penicillins, cephalosporins, carbacephems and carbapenems); aminoglycosides (e.g., sulfonamides, quinolones and the oxazolidinones); glycopeptides (e.g., vancomycin); lincosamides (e.g., clindamycin); lipopeptides; macrolides (e.g., azithromycin); monobactams; nitrofurans; polypeptides (e.g., bacitracin); and tetracyclines.

### C. Use of Medical Polymers Having Sensors to Measure Flow, and Flow Obstruction

As noted above, within various aspects of the present invention medical polymers can be utilized to remove fluid from a patient (e.g., a medical polymer in the form of a drainage catheter); and to provide fluid to a patient (e.g., a medical polymer in the form of a central venous line).

Hence, within one embodiment of the invention polymers are provided (e.g., in the form of a catheter) with one or more sensors that can measure pressure change, and/or fluid flow. They can be utilized to determine whether fluid is draining from the patient, and in certain embodiments to advise a health care provider of impending blockage of the catheter.

Within other embodiments, catheters of the present invention can be utilized to determine whether fluid is flowing into a patient (e.g., in the case of a central venous line), and to determine the proper rate of fluid flow.

### D. Methods for Monitoring Infection in Medical Polymers

Within other embodiments polymers are provided comprising one or more temperature sensors. Such polymers can be utilized to measure the temperature of the polymer, and in the local tissue adjacent to the polymer. Methods are also provided for monitoring changes in temperature over time, in order to determine and/or provide notice (e.g., to a patient and/or healthcare provider) that an infection may be imminent.

In certain embodiments of the present invention, metabolic and physical sensors can also be placed on or within the various components of a polymer in order to monitor for rare, but potentially life-threatening complications of catheters. In some patients, the catheter and surrounding tissues can become infected; typically from bacteria colonizing the patient's own skin that contaminate the surgical field (often Staphylococcus aureus or Staphylococcus epidermidis). Sensors such as temperature sensors (detecting temperature increases), pH sensors (detecting pH decreases), and other metabolic sensors can be used to suggest the presence of infection on or around the implant. For example, temperature sensors may be included within one or more components of a polymer (e.g., in the form of a catheter or mesh) in order to allow early detection of infection could allow preemptive treatment with antibiotics or surgical drainage and eliminate the need to surgically remove the catheter.

Hence, within one embodiment of the invention methods are provided for determining an infection associated with a polymer (e.g., a catheter), comprising the steps of a) providing to a subject a polymer (e.g., catheter, mesh, device or implant) as described herein, wherein the polymer comprises at least one temperature sensor and/or metabolic sensor, and b) detecting a change in said temperature sensor and/or metabolic sensor, and thus determining the presence of an infection. Within various embodiments of the invention the step of detecting may be a series of detections over time, and a change in the sensor is utilized to assess the presence or development of an infection. Within further embodiments a change of 0.5%, 1.0%, or 1.5% elevation of temperature or a metabolic factor over time (e.g., 0.5, 1.0, 1.5, 2.0, 2.5, 3.0, 3.5, 4 hours, 12 hours, 1 day, or 2 days) can be indicative of the presence of an infection (or a developing infection).

Within various embodiments of the invention an antibiotic may be delivered in order to prevent, inhibit or treat an infection subsequent to its detection. Representative examples of suitable antibiotics are well known, and are described above under Section B (the “Therapeutic Agents”)

### E. Further Uses of Sensor-Containing Medical Polymers in Healthcare

Sensors on polymers (e.g., meshes, catheters, endotracheal or chest tubes, bypass grafts, implants and other medical devices), and any associated medical device has a variety of benefits in the healthcare setting, and in non-healthcare settings (e.g., at home or work). For example, postoperative progress can be monitored (readings compared from day-to-day, week-to-week, etc.) and the information compiled and relayed to both the patient and the attending physician allowing rehabilitation to be followed sequentially and compared to expected (typical population) norms. Within certain embodiments, a wearable device interrogates the sensors on a selected or randomized basis, and captures and/or stores the collected sensor data. This data may then be downloaded to another system or device (as described in further detail below).

Integrating the data collected by the sensors described herein (e.g., contact sensors, position sensors, strain gauges and/or accelerometers) with simple, widely available, commercial analytical technologies such as pedometers and global positioning satellite (GPS) capability, allows further clinically important data to be collected such as, but not restricted to: extent of patient ambulation (time, distance, steps, speed, cadence), patient activity levels (frequency of activity, duration, intensity), exercise tolerance (work, calories, power, training effect), range of motion (discussed later) and polymer performance under various “real world” conditions. It is difficult to overstate the value of this information in enabling better management of the patient's recovery. An attending physician (or physiotherapist, rehabilitation specialist) only observes the patient episodically during scheduled visits; the degree of patient function at the exact moment of examination can be impacted by a multitude of disparate factors such as: the presence or absence of pain, the presence or absence of inflammation, time of day, compliance and timing of medication use (pain medications, anti-inflammatories), recent activity, patient strength, mental status, language barriers, the nature of their doctor-patient relationship, or even the patient's ability to accurately articulate their symptoms—to name just a few. Continuous monitoring and data collection can allow the patient and the physician to monitor progress objectively by supplying objective information about patient function under numerous conditions and circumstances, to evaluate how performance has been affected by various interventions (pain control, anti-inflammatory medication, rest, etc.), and to compare patient progress versus previous function and future expected function. Better therapeutic decisions and better patient compliance can be expected when both the doctor and the patient have the benefit of observing the impact of various treatment modalities on patient rehabilitation, activity, function and overall performance.

### F. Generation of Power

Within certain aspects of the invention, a small electrical generation unit can be positioned along an outer, or alternatively an inner, surface of the polymer, or associated delivery device. Briefly, a variety of techniques have been described for scavenging power from small mechanical movements or mechanical vibration. See, for example, the article entitled “Piezoelectric Power Scavenging of Mechanical Vibration Energy,” by U. K. Singh et al., as published in the Australian Mining Technology Conference, Oct. 2-4, 2007, pp. 111-118, and the article entitled “Next Generation Micro-power Systems by Chandrakasan et al., as published in the 2008 Symposium on VLSI Circuits Digest of Technical Papers, pp. 1-5. See also U.S. Pat. No. 8,283,793 entitled “Polymer for Energy Harvesting within a Vessel,” and U.S. Pat. No. 8,311,632 entitled “Polymers, Methods and Systems for Harvesting Energy in the Body,” all of the above of which are incorporated by reference in their entirety. These references provide examples of different types of power scavengers which can produce electricity from very small motion and store the electricity for later use. The above references also describes embodiments in which pressure is applied and released from the particular structure in order to produce electricity without the need for motion, but rather as a result of the application of high pressure. In addition, these references describe embodiments wherein electricity can be produced from pulsatile forces, such as those found within a variety of structures within the body (e.g., within arterial or venous systems).

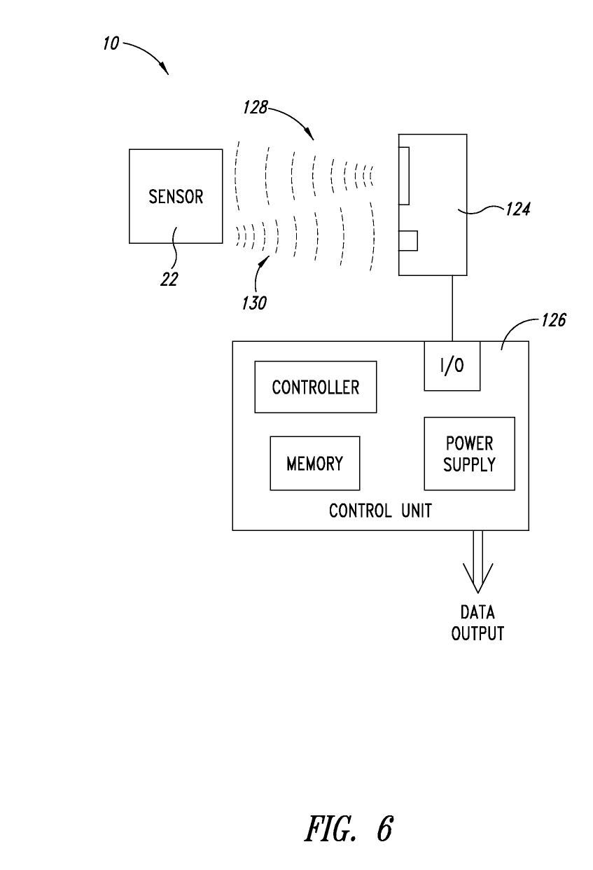

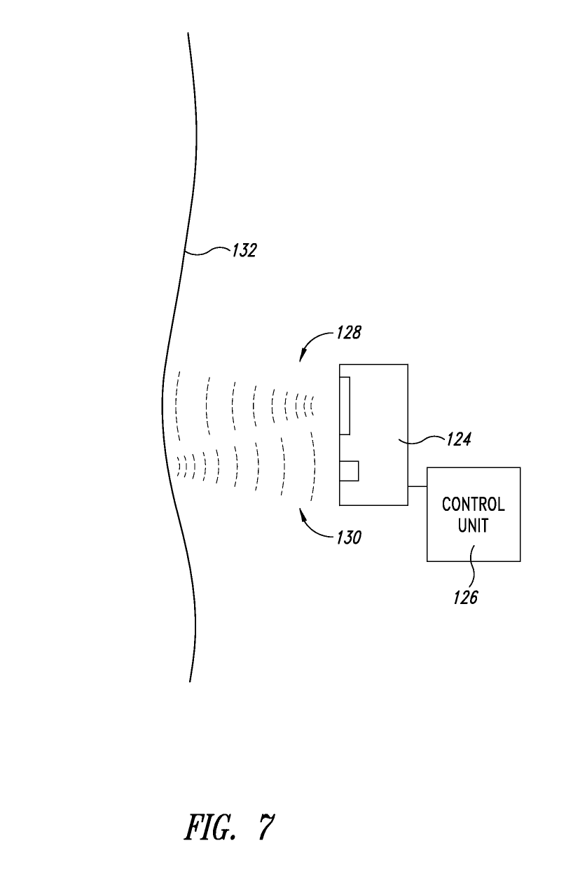

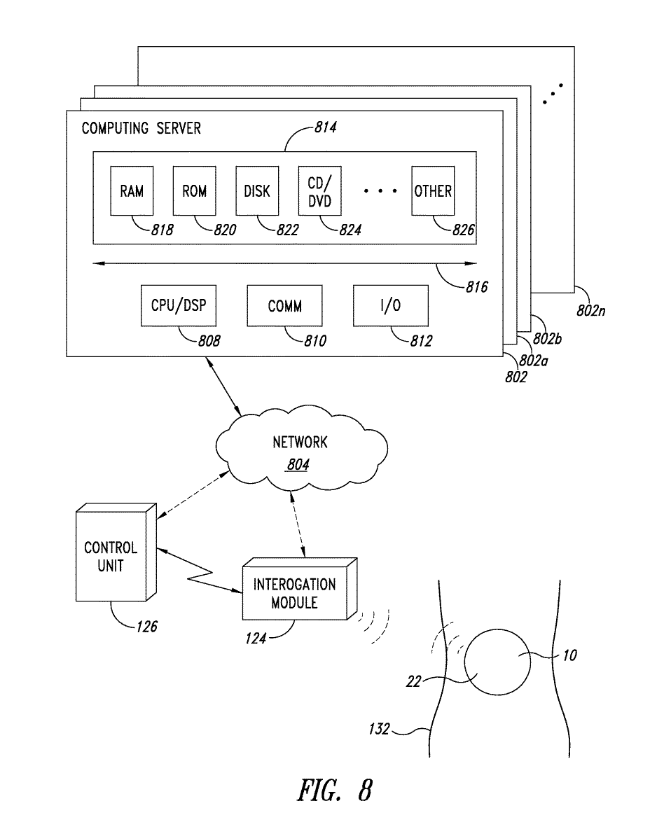

After the electricity is generated by one or more generators, the electricity is transmitted to any one of the variety of sensors which is described herein. For example, it can be transmitted to the sensors 22 shown in FIG. 6, FIG. 7, or FIG. 8 (including for example, contact sensors 22B, position sensors 24, pressure sensors 42 and/or temperature sensors 46). It may also be transmitted to the other sensors described herein. The transmission of the power can be carried out by any acceptable technique. For example, if the sensor is physically coupled to the implant, electric wires may run from the generator to the particular sensor. Alternatively, the electricity can be transmitted wirelessly in the same way that wireless smartcards receive power from closely adjacent power sources using the appropriate send and receive antennas. Such send and receive techniques of electric power are also described in the publication and the patent applications and issued U.S. patent previously described, all of which are incorporated herein by reference.

G. Medical Imaging and Self-Diagnosis of Assemblies Comprising Medical Polymers (e.g., Polymer Containing Hip Prosthesis, Knee Prosthesis, Catheters, Endotracheal or Chest Polymers and Bypass Grafts); Predictive Analysis and Predictive Maintenance

Within other aspects of the invention methods are provided for imaging a polymer and/or associated delivery device, as provided herein, comprising the steps of (a) detecting the location of one or more sensors in a polymer, and/or associated delivery device; and (b) visually displaying the location of said one or more sensors, such that an image of the polymer is created. Within various embodiments, the step of detecting may be done over time, and the visual display may thus show positional movement over time. Within certain preferred embodiments the image which is displayed is a three-dimensional image. Within other embodiment, the imaging techniques may be utilized post-operatively in order to examine the polymer, and/or to compare operation and/or movement of the polymer over time.

The present invention provides polymers and associated delivery devices which are capable of imaging through the use of sensors over a wide variety of conditions. For example, within various aspects of the invention methods are provided for imaging a polymer in the form of a catheter comprising the steps of detecting the changes in sensors in, on, and or within a polymer (e.g., catheter), and wherein the polymer and/or delivery device have sensors at a density of greater than 1, 2, 3, 4, 5, 6, 7, 8, 9, 10 or greater than 10 sensors per square centimeter. Within other aspects the polymer has sensors at a density of greater than 1, 2, 3, 4, 5, 6, 7, 8, 9, 10 or greater than 10 sensors per cubic centimeter. Within either of these embodiments there can be less than 50, 75, 100, or 100 sensors per square centimeter, or per cubic centimeter. Within various embodiments the at least one or more of the sensors may be placed randomly, or at one or more specific locations within the polymer or associated delivery device as described herein. As noted above, a wide variety of sensors can be utilized therein, including for example, contact sensors, strain gauge sensors, pressure sensors, fluid pressure sensors, position sensors, pulse pressure sensors, liquid (e.g., blood) volume sensors, liquid (e.g., blood) flow sensors, liquid (e.g., blood) chemistry sensors, liquid (e.g., blood) metabolic sensors, mechanical stress sensors, and temperature sensors.

For example, a polymer comprising sensors as described herein can be utilized to image anatomy through sensors which can detect positional movement. The sensors used can also include accelerometers and motion sensors to detect movement of the catheter due to a variety of physical changes. Changes in the position of the accelerometers and/or motion sensors over time can be used as a measurement of changes in the position of the catheter over time. Such positional changes can be used as a surrogate marker of anatomy—i.e. they can form an “image” of the polymer in the subject to provide information on the size, shape and location of changes to the polymer, and/or polymer movement/migration.

Certain exemplary embodiments will now be explained in more detail. One particular benefit is the live and in-situ monitoring of the patient's recovery from an implanted polymer (e.g., a polymer containing hip or knee, catheter, mesh, etc.). The sensors as described herein are collecting data on a constant basis, during normal daily activities and even during the night if desired. For example, the contact sensors can obtain and report data once every 10 seconds, once a minute, or once a day. Other sensors will collect data more frequently, such as several times a second. For example, it would be expected that the temperature, contact, and/or position data would be collected and stored several times a second. Other types of data might only need to be collected by the minute or by the hour. Still other sensors may collect data only when signaled by the patient to do so (via an external signaling/triggering polymer) as part of “event recording”—i.e. when the patient experiences a particular event (e.g. pain, injury, instability, etc.)—and signals the sensor containing polymer to obtain a reading at that time in order to allow the comparison of subjective/symptomatic data to objective/sensor data in an effort to better understand the underlying cause or triggers of the patient's symptoms.

In certain instances the polymer (e.g. catheter) is of sufficient size and has more than sufficient space in order to house one or more processor circuits, CPUs, memory chips and other electrical circuits as well as antennas for sending and receiving the data. Within other embodiments, the associated delivery device, or external medical device can be able to house the one or more processor circuits, CPUs, memory c and other electrical circuits as well as antennas for sending and receiving the data. Processors can be programmed to collect data from the various sensors on any desired schedule as set by the medical professional. All activity can be continuously monitored post operation or post-procedure and the data collected and stored in the memory located inside the implant.

A patient will generally have regular medical checkups. When the patient goes to the doctor's office for a medical checkup, the doctor can bring a reading device closely adjacent to the sensor containing polymer (e.g., catheter), in order to transfer the data from the internal circuit inside the implant to the database in the physician's office. The use of wireless transmission using smartcards or other techniques is very well known in the art and need not be described in detail. Examples of such wireless transmission of data are provided in the published patent applications and patents which have been described herein. The data which has been collected (e.g., over a short period of time, over several weeks or even several months) is transferred in a few moments from the memory which is positioned in the implant to the doctor's computer or wireless polymer. The computer therefore analyzes the data for anomalies, unexpected changes over time, positive or negative trends, and other signs which may be indicative of the health of the patient and the operability of the catheter. For example, if the patient has decided to go skiing or jogging, the doctor will be able to monitor the effect of such activity on the sensor containing polymer, including the accelerations and strains during the event itself. The doctor can then look at the health of the catheter in the hours and days after the event and compare it to data prior to the event to determine if any particular event caused long term damage, or if the activities subjected the catheter to forces beyond the manufacturer's performance specifications for that particular sensor containing polymer. Data can be collected and compared with respect to the ongoing and long term performance of the catheter from the strain gauges, the contact sensors, the surface wear sensors, or other sensors which may be present.

In one alternative, the patient may also have such a reading device in their home which collates the data from the sensor containing polymer on a periodic basis, such as once per day or once per week. For example, within certain embodiments the devices and systems provided herein can instruct or otherwise notify the patient, or a permitted third-party as to deviations (e.g., greater than 10%, 20%, 25%, 50%, 70%, and or 100%) from normal, and/or, set parameters. As described above, the patient may also be able to “trigger” a sensor reading (via an external signaling/triggering device) as part of “event recording.” Empowering the patient to follow their own rehabilitation—and enabling them to see the positive (and negative) effects of various lifestyle choices on their health and rehabilitation—can be expected to improve compliance and improve patient outcomes. Furthermore, their experience can be shared via the web with other patients to compare their progress versus expected “norms” for function and rehabilitation and alert them to signs and symptoms that should be brought to their doctor's attention. The performance of different polymer implants can be compared in different patients (different sexes, weights, activity levels, etc.) to help manufacturers design better polymers and assist surgeons and other healthcare providers in the selection of the right implants for specific patient types. Payers, patients, manufacturers and physicians could all benefit from the collection of this comparative information. Lastly, data accumulated at home can be collected and transmitted via the Internet to the physician's office for analysis—potentially eliminating unnecessary visits in some cases and encouraging immediate medical follow-up in others.

### H. Methods of Monitoring Assemblies Comprising Polymers (e.g., Polymer Containing Hip Prosthesis, Knee Prosthesis, Catheters, Endotracheal or Chest Polymers and Bypass Grafts)

As noted above, the present invention also provides methods for monitoring one or more of the sensor containing polymers provided herein. For example, FIG. 6 illustrates a monitoring system usable with a polymer 10 in the form of any one of the Figures described above. The monitoring system includes one or more sensors 22 (including for example, contact sensors 22B, position sensors 24, pressure sensors 42, and/or temperature sensors 46) an interrogation module 124, and a control unit 126. The sensor (e.g., 22, 26, 27 and/or 28) can be passive, wireless type which can operate on power received from a wireless source. Such sensors of this type are well known in the art and widely available. A pressure sensor of this type might be a MEMS pressure sensor, for example, Part No. LPS331AP, sold on the open market by STMicroelectronics. MEMS pressure sensors are well known to operate on very low power and suitable to remain unpowered and idle for long periods of time. They can be provided power wirelessly on an RF signal and, based on the power received wirelessly on the RF signal, perform the pressure sensing and then output the sensed data.

In one embodiment, an electrical generation system (as described above) is provided that can be utilized to power the sensors described herein. During operation, as shown in FIG. 6, an interrogation module 124 outputs a signal 128. The signal 128 is a wireless signal, usually in the RF band, that contains power for the sensors 22 as well as an interrogation request that the sensors perform a sensing. Upon being interrogated with the signal 128, the sensors 22 powers up and stores power in onboard capacitors sufficient to maintain operation during the sensing and data reporting. Such power receiving circuits and storing on onboard capacitors are well known in the art and therefore need not be shown in detail. The appropriate sensing is carried out by the sensors 22 and then the data is output from the sensor back to the interrogation module 124 on a signal 130, where it is received at an input port of the integration module.

According to one embodiment, sufficient signal strength is provided in the initial signal 128 to provide power for the sensor and to carry out the sensing operation and output the signal back to the interrogation module 124. In other embodiments, two or more signals 128 are sent, each signal providing additional power to the sensor to permit it to complete the sensing operation and then provide sufficient power to transfer the data via the signal path 130 back to the interrogation module 124. For example, the signal 128 can be sent continuously, with a sensing request component at the first part of the signal and then continued providing, either as a steady signal or pulses to provide power to operate the sensor. When the sensor is ready to output the data, it sends a signal alerting the interrogation module 124 that data is coming and the signal 128 can be turned off to avoid interference. Alternatively, the integration signal 128 can be at a first frequency and the output signal 130 at a second frequency separated sufficiently that they do not interfere with each other. In a preferred embodiment, they are both the same frequency so that the same antenna on the sensor can receive the signal 128 and send signal 130.

The interrogation signal 128 may contain data to select specific sensors on the catheter. For example, the signal 128 may power up all sensors on the catheter at the same time and then send requests for data from each at different selected times so that with one interrogation signal 128 provided for a set time, such as 1-2 seconds, results in each of the sensors on the catheter collecting data during this time period and then, at the end of the period, reporting the data out on respective signals 130 at different times over the next 0.5 to 2 seconds so that with one interrogation signal 128, the data from all sensors 22 is collected.

The interrogation module 124 is operating under control of the control unit 126 which has a microprocessor for the controller, a memory, an I/O circuit to interface with the interrogation module and a power supply. The control unit may output data to a computer or other device for display and use by the physician to treat the subject.

FIG. 7 illustrates the operation according to a preferred embodiment within a subject. The subject has an outer skin 132. As illustrated in FIG. 7, the interrogation module 124 and control unit 126 are positioned outside the skin 132 of the subject. The interrogation signal 128 passes through the skin of the subject with a wireless RF signal, and the data is received on a wireless RF signal 130 from the sensors within the polymer, back to the interrogation module 124. While the wireless signal can be in any frequency range, an RF range is preferred. A frequency in the VLF to LF ranges of between 3-1300 kHz is preferred to permit the signal to be carried to sufficient depth inside the body with low power, but frequencies below 3 kHz and above 1300 kHz can also be used. The sensing does not require a transfer of large amounts of data and low power is preferred; therefore, a low frequency RF signal is acceptable. This also avoids competition from and inadvertent activation by other wireless signal generators, such as blue tooth, cell phones and the like.

I. Collection, Transmission, Analysis, and Distribution of Data from Assemblies Comprising Polymers (e.g., Polymer Containing Hips, Knees, Meshes, Catheters, Endotracheal or Chest Polymers and Bypass Grafts)

FIG. 8 illustrates one embodiment of an information and communication technology (ICT) system 800 arranged to process sensor data (e.g., data from the sensors 22). In FIG. 8, the ICT system 800 is illustrated to include computing devices that communicate via a network 804, however in other embodiments, the computing devices can communicate directly with each other or through other intervening polymers, and in some cases, the computing devices do not communicate at all. The computing devices of FIG. 8 include computing servers 802, control units 126, interrogation units 124, and other polymers that are not shown for simplicity.

In FIG. 8, one or more sensors 22 communicate with an interrogation module 124. The interrogation module 124 of FIG. 8 is directed by a control unit 126, but in other cases, interrogation modules 124 operates autonomously and passes information to and from sensors 22. One or both of the interrogation module 124 and control unit 126 can communicate with the computing server 802.

Within certain embodiments, the interrogation module and/or the control unit may be a wearable device on the subject. The wearable device (e.g., a watch-like device, a wrist-band, glasses, or other device that may be carried or worn by the subject) can interrogate the sensors over a set (or random) period of time, collect the data, and forward the data on to one or more networks (804). Furthermore, the wearable device may collect data of its own accord which can also be transmitted to the network. Representative examples of data that may be collected include location (e.g., a GPS), body or skin temperature, and other physiologic data (e.g., pulse). Within yet other embodiments, the wearable device may notify the subject directly of any of a number of prescribed conditions, including but not limited to possible or actual failure of the polymer.

The information that is communicated between an interrogation module 124 and the sensors 22, may be useful for many purposes as described herein. In some cases, for example, sensor data information is collected and analyzed expressly for the health of an individual subject. In other cases, sensor data is collected and transmitted to another computing device to be aggregated with other data (for example, the sensor data from 22 may be collected and aggregated with other data collected from a wearable device (e.g., a device that may, in certain embodiments, include GPS data and the like).

FIG. 8 illustrates aspects of a computing server 802 as a cooperative bank of servers further including computing servers 802a, 802b, and one or more other servers 802n. It is understood that computing server 802 may include any number of computing servers that operate individually or collectively to the benefit of users of the computing servers.

In some embodiments, the computing servers 802 are arranged as cloud computing devices created in one or more geographic locations, such as the United States and Canada. The cloud computing devices may be created as MICROSOFT AZURE cloud computing devices or as some other virtually accessible remote computing service.

An interrogation module 124 and a control unit 126 are optionally illustrated as communicating with a computing server 802. Via the interrogation module 124 or control unit 126, sensor data is transferred to (and in addition or alternatively from) a computing server 802 through network 804.

The network 804 includes some or all of cellular communication networks, conventional cable networks, satellite networks, fiber-optic networks, and the like configured as one or more local area networks, wide area networks, personal area networks, and any other type of computing network. In a preferred embodiment, the network 804 includes any communication hardware and software that cooperatively works to permit users of computing devices to view and interact with other computing devices.

Computing server 802 includes a central processing unit (CPU) digital signal processing unit (DSP) 808, communication modules 810, Input/Output (I/O) modules 812, and storage module 814. The components of computing server 802 are cooperatively coupled by one or more buses 816 that facilitate transmission and control of information in and through computing server 802. Communication modules 810 are configurable to pass information between the computer server 802 and other computing devices (e.g., computing servers 802a, 802b, 802n, control unit 126, interrogation unit 124, and the like). I/O modules 812 are configurable to accept input from polymers such as keyboards, computer mice, trackballs, and the like. I/O modules 812 are configurable to provide output to polymers such as displays, recorders, LEDs, audio polymers, and the like.

Storage module 814 may include one or more types of storage media. For example, storage module 814 of FIG. 8 includes random access memory (RAM) 818, read only memory (ROM) 810, disk based memory 822, optical based memory 8124, and other types of memory storage media 8126. In some embodiments one or more memory devices of the storage module 814 has configured thereon one or more database structures. The database structures may be used to store data collected from sensors 22.

In some embodiments, the storage module 814 may further include one or more portions of memory organized a non-transitory computer-readable media (CRM). The CRM is configured to store computing instructions executable by a CPU 808. The computing instructions may be stored as one or more files, and each file may include one or more computer programs. A computer program can be standalone program or part of a larger computer program. Alternatively or in addition, each file may include data or other computational support material for an application that directs the collection, analysis, processing, and/or distribution of data from sensors (e.g., polymer sensors). The sensor data application typically executes a set of instructions stored on computer-readable media.

It will be appreciated that the computing servers shown in the figures and described herein are merely illustrative and are not intended to limit the scope of the present invention. Computing server 802 may be connected to other polymers that are not illustrated, including through one or more networks such as the Internet or via the Web that are incorporated into network 804. More generally, a computing system or polymer (e.g., a “client” or “server”) or any part thereof may comprise any combination of hardware that can interact and perform the described types of functionality, optionally when programmed or otherwise configured with software, including without limitation desktop or other computers, database servers, network storage devices and other network polymers, PDAs, cell phones, glasses, wristbands, wireless phones, pagers, electronic organizers, Internet appliances, television-based systems (e.g., using set-top boxes and/or personal/digital video recorders), and various other products that include appropriate inter-communication capabilities. In addition, the functionality provided by the illustrated system modules may in some embodiments be combined in fewer modules or distributed in additional modules. Similarly, in some embodiments the functionality of some of the illustrated modules may not be provided and/or other additional functionality may be available.

In addition, while various items are illustrated as being stored in memory or on storage while being used, these items or portions of them can be transferred between memory and other storage devices for purposes of memory management and/or data integrity. In at least some embodiments, the illustrated modules and/or systems are software modules/systems that include software instructions which, when executed by the CPU/DSP 808 or other processor, will program the processor to automatically perform the described operations for a module/system. Alternatively, in other embodiments, some or all of the software modules and/or systems may execute in memory on another polymer and communicate with the illustrated computing system/polymer via inter-computer communication.

Furthermore, in some embodiments, some or all of the modules and/or systems may be implemented or provided in other manners, such as at least partially in firmware and/or hardware means, including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic polymers (CPLDs), and the like. Some or all of the systems, modules, or data structures may also be stored (e.g., as software instructions or structured data) on a transitory or non-transitory computer-readable storage medium 814, such as a hard disk 822 or flash drive or other non-volatile storage device 8126, volatile 818 or non-volatile memory 810, a network storage device, or a portable media article (e.g., a DVD disk, a CD disk, an optical disk, a flash memory device, etc.) to be read by an appropriate input or output system or via an appropriate connection. The systems, modules, and data structures may also in some embodiments be transmitted as generated data signals (e.g., as part of a carrier wave or other analog or digital propagated signal) on a variety of computer readable transmission mediums, including wireless-based and wired/cable-based mediums. The data signals can take a variety of forms such as part of a single or multiplexed analog signal, as multiple discrete digital packets or frames, as a discrete or streaming set of digital bits, or in some other form. Such computer program products may also take other forms in other embodiments. Accordingly, the present invention may be practiced with other computer system configurations.

In FIG. 8, sensor data from, e.g., sensors 22 is provided to computing server 802. Generally speaking, the sensor data represents data retrieved from a known subject and from a known sensor. The sensor data may possess include or be further associated with additional information such as the USI, UDI, a time stamp, a location (e.g., GPS) stamp, a date stamp, and other information. The differences between various sensors is that some may include more or fewer data bits that associate the data with a particular source, collection polymer, transmission characteristic, or the like.

In some embodiments, the sensor data may comprise sensitive information such as private health information associated with a specific subject. Sensitive information, for example sensor data from sensors e.g., 22, may include any information that an associated party desires to keep from wide or easy dissemination. Sensitive information can stand alone or be combined with other non-sensitive information. For example, a subject's medical information is typically sensitive information. In some cases, the storage and transmission of a subject's medical information is protected by a government directive (e.g., law, regulation, etc.) such as the U.S. Health Insurance Portability and Accountability Act (HIPPA).

As discussed herein, a reference to “sensitive” information includes information that is entirely sensitive and information that is some combination of sensitive and non-sensitive information. The sensitive information may be represented in a data file or in some other format. As used herein, a data file that includes a subject's medical information may be referred to as “sensitive information.” Other information, such as employment information, financial information, identity information, and many other types of information may also be considered sensitive information.

A computing system can represent sensitive information with an encoding algorithm (e.g., ASCII), a well-recognized file format (e.g., PDF), or by some other format. In a computing system, sensitive information can be protected from wide or easy dissemination with an encryption algorithm.

Generally speaking, sensitive information can be stored by a computing system as a discrete set of data bits. The set of data bits may be called “plaintext.” Furthermore, a computing system can use an encryption process to transform plaintext using an encryption algorithm (i.e., a cipher) into a set of data bits having a highly unreadable state (i.e., cipher text). A computing system having knowledge of the encryption key used to create the cipher text can restore the information to a plaintext readable state. Accordingly, in some cases, sensitive data (e.g., sensor data 806a, 806b) is optionally encrypted before being communicated to a computing device.

In one embodiment, the operation of the information and communication technology (ICT) system 800 of FIG. 8 includes one or more sensor data computer programs stored on a computer-readable medium. The computer program may optionally direct and/or receive data from one or more catheter sensors implanted in one or more subjects. A sensor data computer program may be executed in a computing server 802. Alternatively, or in addition, a sensor data computer program may be executed in a control unit 126, an interrogation unit 124.

In one embodiment, a computer program to direct the collection and use of catheter sensor data is stored on a non-transitory computer-readable medium in storage module 814. The computer program is configured to identify a subject who has a wireless catheter inserted in his or her body. The wireless polymer may include one or more wireless sensors.

In some cases, the computer program identifies one subject, and in other cases, two or more subjects are identified. The subjects may each have one or more polymers containing wireless sensors (e.g., polymer containing hip prosthesis, knee prosthesis, catheters, endotracheal or chest polymers and bypass grafts), and each wireless device may have one or more wireless sensors of the type described herein.

The computer program is arranged to direct the collection of sensor data from the wireless catheter polymers. The sensor data is generally collected with a wireless interrogation unit 124. In some cases, the program communicates with the wireless interrogation unit 124. In other cases, the program communicates with a control unit 126, which in turn directs a wireless interrogation unit 124. In still other cases, some other mechanism is used direct the collection of the sensor data.

Once the sensor data is collected, the data may be further processed. For example, in some cases, the sensor data includes sensitive subject data, which can be removed or disassociated with the data. The sensor data can be individually stored (e.g., by unique sensor identification number, polymer number, etc.) or aggregated together with other sensor data by sensor type, time stamp, location stamp, date stamp, subject type, other subject characteristics, or by some other means.

The following pseudo-code description is used to generally illustrate one exemplary algorithm executed by a computing server 802 and generally described herein with respect to FIG. 8:

Start
Open a secure socket layer (SSL)
Identify a subject
Communicate with a predetermined control unit
Request sensor data from the subject via the control unit
Receive sensor data
If the sensor data is encrypted

THEN decrypt the sensor data

Store encrypted data in the selected storage locations
Aggregate the sensor data with other sensor data
Store encrypted data in the selected storage locations
Maintain a record of the storage transaction
Perform post storage actions
End

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., AT&T, T-Mobile, Verizon), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

In conclusion, polymers having a variety of sensors can be utilized to serve a variety of critical clinical functions, such as safe, accurate and less traumatic placement and deployment of a polymer containing medical device (e.g., a catheter), procedural and post-operative “real time” imaging of the polymer and the surrounding anatomy, the development of complications associated with the polymer, and the patient's overall health status. Currently, post-operative (both in hospital and out-patient) evaluation of medical devices (e.g., catheters) in patients is through patient history, physical examination and medical monitoring that is supplemented with diagnostic imaging studies as required. However, most of the patient's recuperative period occurs between hospital and office visits and the majority of data on daily function goes uncaptured; furthermore, monitoring patient progress through the use of some diagnostic imaging technology can be expensive, invasive and carry its own health risks (the use of nuclear isotopes or certain dyes). It can, therefore, be very difficult to accurately measure and follow the development or worsening of symptoms and evaluate “real life” catheter performance, particularly as they relate to patient activity levels, exercise tolerance, and the effectiveness of rehabilitation efforts and medications.

At present, neither the physician nor the patient has access to the type of “real time,” continuous, objective, catheter performance measurements that they might otherwise like to have. Being able to monitor in situ polymer function, integrity, anatomy and physiology can provide the physician with valuable objective information during office visits; furthermore, the patient can take additional readings at home at various times (e.g. when experiencing pain, during exercise, after taking medications, etc.) to provide important complementary clinical information to the doctor (which can be sent to the healthcare provider electronically even from remote locations). From the perspective of the patient, being able to monitor many of these same parameters at home allows them to take a more proactive role in their care and recovery and provide him or her with either an early warning indicator to seek medical assistance or with reassurance.

In one alternative, the patient may have a reading device in their home which collates the data from the catheter on a periodic basis, such as once per day or once per week. In addition to empowering the patient to follow their own rehabilitation—and enabling them to see the positive (and negative) effects of various lifestyle choices on their health and rehabilitation—such information access can be expected to improve compliance and improve patient outcomes. For example, within certain embodiments the polymers and related systems provided herein can instruct and/or notify the patient, or a permitted third-party as to deviations (e.g., greater than 10%, 20%, 25%, 50%, 70%, and or 100%) from normal, and/or, set parameters. Furthermore, their recovery experience can be shared via the web with other patients to compare their progress versus expected “norms” for function and rehabilitation and alert them to signs and symptoms that should be brought to their doctor's attention. From a public health perspective, the performance of different polymers can be compared in different patients (different sexes, disease severity, activity levels, concurrent diseases such as hypertension and diabetes, smoking status, obesity, etc.) to help manufacturers design better polymers and assist physicians in the selection of the right polymer or polymeric device for specific patient types. Payers, patients, manufacturers and physicians could all benefit from the collection of this comparative information. Poor and dangerous products could be identified and removed from the market and objective long-term effectiveness data collected and analyzed. Lastly, data accumulated at home can be collected and transmitted via the Internet to the physician's office for analysis—potentially eliminating unnecessary visits in some cases and encouraging immediate medical follow-up in others.

### Conventions

In general, and unless otherwise specified, all technical and scientific terms used herein shall have the same meaning as those commonly understood by one of ordinary skill in the art to which the embodiment pertains. For convenience, the meanings of selected terms are provided below, where these meanings are provided in order to aid in describing embodiments identified herein. Unless stated otherwise, or unless implicit from the context in which the term is used, the meanings provided below are the meanings intended for the referenced term.

Embodiment examples or feature examples specifically provided are intended to be exemplary only, that is, those examples are non-limiting on an embodiment. The term “e.g.” (Latin, exempli gratia) is used herein to refer to a non-limiting example, and effectively means “for example”.

Singular terms shall include pluralities and plural terms shall include the singular, unless otherwise specified or required by context. For example, the singular terms “a”, “an” and “the” include plural referents unless the context clearly indicates otherwise. Similarly, the term “or” is intended to include “and” unless the context clearly indicates otherwise.

Except in specific examples provided herein, or where otherwise indicated, all numbers expressing quantities of a component should be understood as modified in all instances by the term “about”, where “about” means±5% of the stated value, e.g., 100 refers to any value within the range of 95-105.

The terms comprise, comprising and comprises are used to identify essential features of an embodiment, where the embodiment may be, for example, a composition, polymer, method or kit. The embodiment may optionally contain one or more additional unspecified features, and so the term comprises may be understood to mean includes.

The following are some specific numbered embodiments of the systems and processes disclosed herein. These embodiments are exemplary only. It will be understood that the invention is not limited to the embodiments set forth herein for illustration, but embraces all such forms thereof as come within the scope of the above disclosure.

1) A medical polymer comprising:

a medical polymer and one or more sensors positioned within or upon said medical polymer.

2) The medical polymer of embodiment 1 wherein said one or more sensors includes a sensor within the matrix of the medical polymer.

3) The medical polymer of embodiment 1 wherein said one or more sensors includes a sensor within or upon said medical polymer.

4) The medical polymer according to any one of embodiments 1 to 4 wherein said sensor is selected from the group consisting of fluid pressure sensors, contact sensors, position sensors, pulse pressure sensors, liquid volume sensors, liquid flow sensors, chemistry sensors, metabolic sensors, accelerometers, mechanical stress sensors and temperature sensors.

5) The medical polymer according to embodiment 1 wherein said medical polymer is a biodegradable polymer.

6) The medical polymer according to embodiment 5 wherein said biodegradable polymer is collagen, HA, PLA, or PGLA.

7) The medical polymer according to embodiment 1 wherein said medical polymer is a non-biodegradable polymer.

8) The medical polymer according to embodiment 7 wherein said non-biodegradable polymer is silicone, polyurethane, PTFE, PMMA, or PEEK.

9) The medical polymer according to any one of embodiments 1 to 8 wherein said sensor is selected from the group consisting of accelerometers, pressure sensors, contact sensors, position sensors, chemical microsensors, tissue metabolic sensors, mechanical stress sensors and temperature sensors.

10) The medical polymer according to embodiment 9 wherein said accelerometer detects acceleration, tilt, vibration, shock and or rotation.

11) The medical polymer according to any one of embodiments 1 to 10 further comprising:

an electronic processor positioned upon and/or inside the medical polymer that is electrically coupled to sensors.

12) The medical polymer according to embodiment 11 wherein the electric coupling is a wireless coupling.

13) The medical polymer according to embodiment 11 further including:

a memory coupled to the electronic processor and positioned upon and/or inside the medical polymer.

14) A medical polymer according to any one of embodiments 1 to 13 formed into a solid form.

15) A medical polymer according to any one of embodiments 1 to 13 formed into a liquid.

16) The medical polymer according to any one of embodiments 1 to 15 wherein said sensor is a plurality of sensors which are positioned on or within said polymer, medical polymer and/or kit at a density of greater than 1, 2, 3, 4, 5, 6, 7, 8, 9, 10 or 20 sensors per square centimeter.

17) The medical polymer according to any one of embodiments 1 to 15 wherein said sensor is a plurality of sensors which are positioned on or within said polymer, medical polymer and/or kit at a density of greater than 1, 2, 3, 4, 5, 6, 7, 8, 9, 10 or 20 sensors per cubic centimeter.

18) The medical polymer according to any one of embodiments 1 to 17 wherein said sensors are placed randomly within the medical polymer.

19) The medical polymer according to any one of embodiments 1 to 17 wherein the one or more of the sensors are placed at specific locations within the medical polymer.

20) A method comprising:

obtaining data from a sensor positioned at a plurality of locations between on and/or within a medical polymer according to any one of embodiments 1 to 19 of a subject;

storing the data in a memory device located on or within the medical polymer; and

transferring the data from the memory to a location outside the polymer or medical polymer.

21) A method according to embodiment 20, further comprising the step of analyzing said data.

22) A method for detecting and/or recording an event in a subject with a medical polymer as provided in any one of embodiments 1 to 19, comprising the step of interrogating at a desired point in time the activity of one or more sensors within the medical polymer, and recording said activity.

23) The method according to embodiment 22 wherein the step of interrogating is performed by a subject which has an implanted polymer, and the step of recording is performed on a wearable device.

24) The method according to any one of embodiments 22, or 23, wherein said recording is provided to a health care provider.

25) A method for imaging a medical polymer, comprising the steps of
(a) detecting the location of one or more sensors of a medical polymer according to any one of embodiments 1 to 19; and
(b) visually displaying the location of said one or more sensors, such that an image of the medical polymer is created.

26) The method according to embodiment 25 wherein the step of detecting occurs over time.

27) The method according to embodiment 25 or 26, wherein said visual display shows changes in the positions of said sensors over time, and/or changes in temperature of the sensors or surrounding tissue over time.

28) The method according to any one of embodiments 25 to 27 wherein said visual display is a three-dimensional image of said polymer.

29) A method for inserting a medical polymer into a subject, comprising the steps of

(a) inserting a medical polymer according to any one of embodiments 1 to 19 into a subject; and

(b) imaging the placement of said medical polymer according to the method of any one of embodiments 25 to 28.

30) A method for examining a medical polymer according to any one of embodiments 1 to 19 which has been previously inserted into a patient, comprising the step of imaging the polymer according to the method of any one of embodiments 25 to 28.

31) A method of monitoring a medical polymer within a subject, comprising:

transmitting a wireless electrical signal from a location outside the body to a location inside the subject's body;

receiving the signal at a sensor positioned on a medical polymer according to any one of embodiments 1 to 19 located inside the body;

powering the sensor using the received signal;

sensing data at the sensor; and

outputting the sensed data from the sensor to a receiving unit located outside of the body.

32) The method according to embodiment 31 wherein said receiving unit is a watch, wrist band, cell phone or glasses.

33) The method according to embodiments 31 or 32 wherein said receiving unit is located within a subject's residence or office.

34) The method according to embodiments any one of embodiments 31 to 33 wherein said sensed data is provided to a health care provider.

35) The method according to any one of embodiments 31 to 34 wherein said sensed data is posted to one or more websites.

36) A non-transitory computer-readable storage medium whose stored contents configure a computing system to perform a method, the method comprising:

identifying a subject, the identified subject having at least one wireless medical polymer according to any one of embodiments 1 to 19, each wireless medical polymer having one or more wireless sensors;

directing a wireless interrogation unit to collect sensor data from at least one of the respective one or more wireless sensors; and

receiving the collected sensor data.

37) The non-transitory computer-readable storage medium of embodiment 36 whose stored contents configure a computing system to perform a method, the method further comprising:

identifying a plurality of subjects, each identified subject having at least one wireless polymer, medical polymer, or kit, each wireless medical polymer having one or more wireless sensors;

directing a wireless interrogation unit associated with each identified subject to collect sensor data from at least one of the respective one or more wireless sensors;

receiving the collected sensor data; and

aggregating the collected sensor data.

38) The non-transitory computer-readable storage medium of embodiment 36 whose stored contents configure a computing system to perform a method, the method further comprising:

removing sensitive subject data from the collected sensor data; and

parsing the aggregated data according to a type of sensor.

39) The non-transitory computer-readable storage medium of embodiment 36 whose stored contents configure a computing system to perform a method, wherein directing the wireless interrogation unit includes directing a control unit associated with the wireless interrogation unit.

40) The non-transitory computer readable storage medium according to any one of embodiments 36 to 39, wherein said medical polymer is an assembly according to any one of embodiments 1 to 19.

41) The storage medium according to any one of embodiments 36 to 40 wherein said collected sensor data is received on a watch, wrist band, cell phone or glasses.

42) The storage medium according to any one of embodiments 36 to 41 wherein said collected sensor data is received within a subject's residence or office.

43) The storage medium according to any one of embodiments 36 to 42 wherein said collected sensed data is provided to a health care provider.

44) The storage medium according to any one of embodiments 36 to 43 wherein said sensed data is posted to one or more websites.

45) The method according to any one of embodiments 31 to 35, or storage medium according to any one of embodiments 36 to 44, wherein said data is analyzed.

46) The method or storage medium according to embodiment 45 wherein said data is plotted to enable visualization of change over time.

47) The method or storage medium according to embodiments 45 or 46 wherein said data is plotted to provide a three-dimensional image.

48) A method for determining degradation of a polymer, comprising the steps of a) providing to a subject a polymer according to any one of embodiments 1 to 19, and b) detecting a change in a sensor, and thus determining degradation of the polymer.

49) The method according to embodiment 48 wherein said sensor is capable of detecting one or more physiological and/or locational parameters.

50) The method according to embodiment 48 or 49 wherein said sensor detects contact, fluid flow, pressure and/or temperature.

51) The method according to any one of embodiments 48 to 50 wherein said sensor detects a location within the subject.

52) The method according to any one of embodiments 48 to 50 wherein said sensor moves and/or is eliminated by the body upon degradation of the polymer.

53) The method according to any one of embodiments 48 to 52 wherein the step of detecting is a series of detections over time.

54) A method for determining an infection associated with a polymer, comprising the steps of a) providing to a subject a polymer according to any one of embodiments 1 to 19, wherein said polymer comprises at least one temperature sensor and/or metabolic sensor, and b) detecting a change in said temperature sensor and/or metabolic sensor, and thus determining the presence of an infection.

55) The method according to embodiment 54 wherein the step of detecting is a series of detections over time.

56) The method according to embodiments 54 or 55 wherein said change is greater than a 1% change over the period of one hour.

57) The method according to embodiments 54 to 56 wherein said change is a continually increasing temperature and/or metabolic activity over the course of 4 hours.

The various embodiments described above can be combined to provide further embodiments. All of the U.S. patents, U.S. patent application publications, U.S. patent applications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A medical polymer comprising:
a medical polymer and one or more sensors positioned within or upon said medical polymer.

2. The medical polymer of claim 1 wherein said one or more sensors includes a sensor within the matrix of the medical polymer.

3. The medical polymer of claim 1 wherein said one or more sensors includes a sensor within or upon said medical polymer.

4. The medical polymer according to any one of claims 1 to 4 wherein said sensor is selected from the group consisting of fluid pressure sensors, contact sensors, position sensors, pulse pressure sensors, liquid volume sensors, liquid flow sensors, chemistry sensors, metabolic sensors, accelerometers, mechanical stress sensors and temperature sensors.

5. The medical polymer according to claim 1 wherein said medical polymer is a biodegradable polymer.

6. The medical polymer according to claim 5 wherein said biodegradable polymer is collagen, HA, PLA, or PGLA.

7. The medical polymer according to claim 1 wherein said medical polymer is a non-biodegradable polymer.

8. The medical polymer according to claim 7 wherein said non-biodegradable polymer is silicone, polyurethane, PTFE, PMMA, or PEEK.

9. The medical polymer according to any one of claims 1 to 8 wherein said sensor is selected from the group consisting of accelerometers, pressure sensors, contact sensors, position sensors, chemical microsensors, tissue metabolic sensors, mechanical stress sensors and temperature sensors.

10. The medical polymer according to claim 9 wherein said accelerometer detects acceleration, tilt, vibration, shock and or rotation.

11. The medical polymer according to any one of claims 1 to 10 further comprising:
an electronic processor positioned upon and/or inside the medical polymer that is electrically coupled to sensors.

12. The medical polymer according to claim 11 wherein the electric coupling is a wireless coupling.

13. The medical polymer according to claim 11 further including:
a memory coupled to the electronic processor and positioned upon and/or inside the medical polymer.

14. A medical polymer according to any one of claims 1 to 13 formed into a solid form.

15. A medical polymer according to any one of claims 1 to 13 formed into a liquid.

16. The medical polymer according to any one of claims 1 to 15 wherein said sensor is a plurality of sensors which are positioned on or within said polymer, medical polymer and/or kit at a density of greater than 1, 2, 3, 4, 5, 6, 7, 8, 9, 10 or 20 sensors per square centimeter.

17. The medical polymer according to any one of claims 1 to 15 wherein said sensor is a plurality of sensors which are positioned on or within said polymer, medical polymer and/or kit at a density of greater than 1, 2, 3, 4, 5, 6, 7, 8, 9, 10 or 20 sensors per cubic centimeter.

18. The medical polymer according to any one of claims 1 to 17 wherein said sensors are placed randomly within the medical polymer.

19. The medical polymer according to any one of claims 1 to 17 wherein the one or more of the sensors are placed at specific locations within the medical polymer.

20. A method comprising:
obtaining data from a sensor positioned at a plurality of locations between on and/or within a medical polymer according to any one of claims 1 to 19 of a subject;
storing the data in a memory device located on or within the medical polymer; and
transferring the data from the memory to a location outside the polymer or medical polymer.

21. A method according to claim 20, further comprising the step of analyzing said data.

22. A method for detecting and/or recording an event in a subject with a medical polymer as provided in any one of claims 1 to 19, comprising the step of interrogating at a desired point in time the activity of one or more sensors within the medical polymer, and recording said activity.

23. The method according to claim 22 wherein the step of interrogating is performed by a subject which has an implanted polymer, and the step of recording is performed on a wearable device.

24. The method according to any one of claim 22, or 23, wherein said recording is provided to a health care provider.

25. A method for imaging a medical polymer, comprising the steps of
(a) detecting the location of one or more sensors of a medical polymer according to any one of claims 1 to 19; and
(b) visually displaying the location of said one or more sensors, such that an image of the medical polymer is created.

26. The method according to claim 25 wherein the step of detecting occurs over time.

27. The method according to claim 25 or 26, wherein said visual display shows changes in the positions of said sensors over time, and/or changes in temperature of the sensors or surrounding tissue over time.

28. The method according to any one of claims 25 to 27 wherein said visual display is a three-dimensional image of said polymer.

29. A method for inserting a medical polymer into a subject, comprising the steps of
(a) inserting a medical polymer according to any one of claims 1 to 19 into a subject; and
(b) imaging the placement of said medical polymer according to the method of any one of claims 25 to 28.

30. A method for examining a medical polymer according to any one of claims 1 to 19 which has been previously inserted into a patient, comprising the step of imaging the polymer according to the method of any one of claims 25 to 28.

31. A method of monitoring a medical polymer within a subject, comprising:
transmitting a wireless electrical signal from a location outside the body to a location inside the subject's body;
receiving the signal at a sensor positioned on a medical polymer according to any one of claims 1 to 19 located inside the body;
powering the sensor using the received signal;
sensing data at the sensor; and
outputting the sensed data from the sensor to a receiving unit located outside of the body.

32. The method according to claim 31 wherein said receiving unit is a watch, wrist band, cell phone or glasses.

33. The method according to claim 31 or 32 wherein said receiving unit is located within a subject's residence or office.

34. The method according to claims any one of claims 31 to 33 wherein said sensed data is provided to a health care provider.

35. The method according to any one of claims 31 to 34 wherein said sensed data is posted to one or more websites.

36. A non-transitory computer-readable storage medium whose stored contents configure a computing system to perform a method, the method comprising:
identifying a subject, the identified subject having at least one wireless medical polymer according to any one of claims 1 to 19, each wireless medical polymer having one or more wireless sensors;
directing a wireless interrogation unit to collect sensor data from at least one of the respective one or more wireless sensors; and
receiving the collected sensor data.

37. The non-transitory computer-readable storage medium of claim 36 whose stored contents configure a computing system to perform a method, the method further comprising:
identifying a plurality of subjects, each identified subject having at least one wireless polymer, medical polymer, or kit, each wireless medical polymer having one or more wireless sensors;
directing a wireless interrogation unit associated with each identified subject to collect sensor data from at least one of the respective one or more wireless sensors;
receiving the collected sensor data; and
aggregating the collected sensor data.

38. The non-transitory computer-readable storage medium of claim 36 whose stored contents configure a computing system to perform a method, the method further comprising:
removing sensitive subject data from the collected sensor data; and
parsing the aggregated data according to a type of sensor.

39. The non-transitory computer-readable storage medium of claim 36 whose stored contents configure a computing system to perform a method, wherein directing the wireless interrogation unit includes directing a control unit associated with the wireless interrogation unit.

40. The non-transitory computer readable storage medium according to any one of claims 36 to 39, wherein said medical polymer is an assembly according to any one of claims 1 to 19.

41. The storage medium according to any one of claims 36 to 40 wherein said collected sensor data is received on a watch, wrist band, cell phone or glasses.

42. The storage medium according to any one of claims 36 to 41 wherein said collected sensor data is received within a subject's residence or office.

43. The storage medium according to any one of claims 36 to 42 wherein said collected sensed data is provided to a health care provider.

44. The storage medium according to any one of claims 36 to 43 wherein said sensed data is posted to one or more websites.

45. The method according to any one of claims 31 to 35, or storage medium according to any one of claims 36 to 44, wherein said data is analyzed.

46. The method or storage medium according to claim 45 wherein said data is plotted to enable visualization of change over time.

47. The method or storage medium according to claim 45 or 46 wherein said data is plotted to provide a three-dimensional image.

48. A method for determining degradation of a polymer, comprising the steps of a) providing to a subject a polymer according to any one of claims 1 to 19, and b) detecting a change in a sensor, and thus determining degradation of the polymer.

49. The method according to claim 48 wherein said sensor is capable of detecting one or more physiological and/or locational parameters.

50. The method according to claim 48 or 49 wherein said sensor detects contact, fluid flow, pressure and/or temperature.

51. The method according to any one of claims 48 to 50 wherein said sensor detects a location within the subject.

52. The method according to any one of claims 48 to 50 wherein said sensor moves and/or is eliminated by the body upon degradation of the polymer.

53. The method according to any one of claims 48 to 52 wherein the step of detecting is a series of detections over time.

54. A method for determining an infection associated with a polymer, comprising the steps of a) providing to a subject a polymer according to any one of claims 1 to 19, wherein said polymer comprises at least one temperature sensor and/or metabolic sensor, and b) detecting a change in said temperature sensor and/or metabolic sensor, and thus determining the presence of an infection.

55. The method according to claim 54 wherein the step of detecting is a series of detections over time.

56. The method according to claim 54 or 55 wherein said change is greater than a 1% change over the period of one hour.

57. The method according to claims 54 to 56 wherein said change is a continually increasing temperature and/or metabolic activity over the course of 4 hours.
